SEQUENCE LISTING (4 sequences)

SEQ ID NO: 1 - DNA, 16 bp, Artificial
  LNA oligonucleotide
gagttacttgccaact

SEQ ID NO: 2 - DNA, 13 bp, Artificial
  LNA Oligonucleotide
gcattggtattca

SEQ ID NO: 3 - DNA, 13 bp, Artificial
  LNA Oligonucleotide
acttatggttacg

SEQ ID NO: 4 - DNA, 16 bp, Artificial
  LNA Oligonucleotide
cacattccttgctctg

(19) United States
(12) Patent Application Publication
(10) Pub. No.: US 2021/0309690 A1
(43) Pub. Date: Oct. 7, 2021

(54) OLIGONUCLEOTIDE SYNTHESIS

(71) Applicant: ROCHE INNOVATION CENTER COPENHAGEN A/S, Horsholm (DK)

(72) Inventors: Konrad Bleicher, Basel (CH); Joerg Duschmalé, Basel (CH); Goutam Saha, Salt Lake, Kolkata (IN); Juber Abdulhamid Shaikh, Hinjewadi MIDC (IN)

(21) Appl. No.: 16/320,918

(22) PCT Filed: Jul. 25, 2017
(86) PCT No.: PCT/EP2017/068700

§ 371 Date: Jan. 25, 2019

(30) Foreign Application Priority Data
Jul. 27, 2016 (IN) 201641025647

(51) Int. Cl. C07H 21/00

(52) U.S. Cl. CPC C07H 21/00

(57) ABSTRACT

The invention relates to a process for the manufacture of an oligonucleotide comprising at least one non-chiral phosphorothioate intemucleoside linkage of formula (I) wherein R¹ is as defined in the description and in the claims.

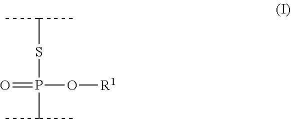

The invention relates to a process for the manufacture of oligonucleotides, in particular of oligonucleotides comprising a phosphorothioate linkage, in particular a non-chiral phosphorothioate linkage.

The invention relates in particular to a process for the manufacture of an oligonucleotide comprising at least one non-chiral phosphorothioate internucleoside linkage of formula (I)

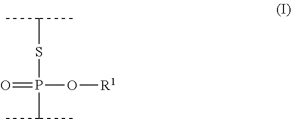

said process comprising the step of reacting an oligonucleotide comprising an internucleoside linkage of formula (II)

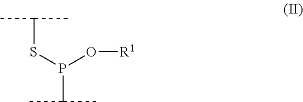

in the presence of iodine, wherein the concentration of iodine is between about 0.001 M and about 0.01 M; and
wherein R¹ is a phosphate protecting group.

The use of oligodeoxynucleotides as therapeutic agents, where the well-understood principles of Watson-Crick hybridization are exploited to target complementary RNA strands, has witnessed remarkable progress since its inception in the late 1970's (P. C. Zamecnik, M. L. Stephenson, P Natl Acad Sci USA 1978, 75, 280-284; S. T. Crooke, Antisense drug technology: principles, strategies, and applications, 2nd ed. ed., Boca Raton, Fla.: CRC Press, 2008).

Several types of chemical modifications have been introduced over time in synthetic oligonucleotides in order to e.g. extend their half-live, improve pharmacokinetics, enhance the RNaseH activity, reduce toxicity or enhance mismatch discrimination.

One of the most successful modifications is the introduction of phosphorothioate linkages, where one of the nonbridging phosphate oxygen atoms is replaced with a sulfur atom (F. Eckstein, Antisense and Nucleic Acid Drug Development 2009, 10, 117-121). Such phosphorothioate oligodeoxynucleotides show an increased protein binding as well as a distinctly higher stability to nucleolytic degradation and thus a substantially higher half-live in plasma, tissues and cells than their unmodified phosphodiester analogues. This allowed the development of the first generation of oligonucleotide therapeutics and opened the door of the later generation modifications such as Locked Nucleic Acids (LNAs).

Replacement of a phosphodiester linkage with a phosphorothioate, however, creates a chiral center at the phosphorous atom. As a consequence, all approved oligonucleotide therapeutics until now are mixtures of a huge amount of diastereoisomeric compounds, with potentially different (and possibly opposing) physiochemical properties.

In order to reduce the diastereoisomeric complexity of such oligodeoxynucleotides, the sulfur atom within a phosphorothioate can in theory be shifted from one of the nonbridging positions to the bridging 3′-position of the ribose sugar. This modification renders the substitution pattern around the phosphorous symmetrical and thus removes the chiral center, consequently reducing the diastereoisomeric complexity of the molecule. While such 3′-deoxy-3′-mercapto- as well as 2′,3′-dideoxy-3′-mercaptonucleotides (M. M. Piperakis, J. W. Gaynor, J. Fisher, R. Cosstick, Org. Biomol. Chem. 2013, 11, 966-974; J. Bentley, J. A. Brazier, J. Fisher, R. Cosstick, Org. Biomol. Chem. 2007, 5, 3698-3702; G. Sabbagh, K. J. Fettes, R. Gosain, I. A. O'Neil, R. Cosstick, Nucleic Acids Res. 2004, 32, 495-501; A. P. G. Beevers, K. J. Fettes, G. Sabbagh, F. K. Murad, J. R. P. Arnold, R. Cosstick, J. Fisher, Org. Biomol. Chem. 2004, 2, 114-119) have previously been introduced into oligo(deoxy)nucleotides, this modification has neither been described in the context of therapeutics, nor with the intention to reduce the diastereoisomeric complexity of oligodeoxynucleotide phosphorothioates.

The synthesis of longer oligodeoxynucleotides bearing one or more modified 2′,3′-dideoxy-3′-mercaptonucleotides is generally performed by solid phase oligonucleotide synthesis techniques involving 3′-mercapto phosphoramidite building blocks (see above references). However, when using such modified phosphoramidites, low coupling efficiencies are reported, resulting in low yields of the desired products. Previous optimization efforts have therefore generally focused on the coupling conditions (reagent concentration, coupling time, activators or additives) but have not delivered a universal set of conditions dramatically improving synthetic efficiency.

We verified that the incorporation of 2′-3′-dideoxy-3′-mercapto-phosphoramidites into longer model oligodeoxynucleotides was difficult. Furthermore, we also found the subsequent oxidation surprisingly difficult. This contributed to a significant extent to the observed low synthetic efficiency. Under standard conditions, rather highly concentrated solutions of iodine are generally used as the oxidant (often as high as 0.1M). However, under these conditions a deletion fragment bearing an additional phosphate group at the 5′-end of the oligonucleotide is generally observed as a byproduct (often in equal amounts to the desired product).

In contrast, it was surprisingly found that using iodine as the oxidant in very low concentrations (generally at least a factor 50 lower than under standard conditions known in the art), the formation of the competing byproduct could be suppressed to a considerable extent. The application of these new oxidation conditions now allows the efficient incorporation of the 2′,3′-dideoxy-3′-mercapto modification into oligonucleotides.

It is understood that the phosphorothioate internucleoside linkage of formula (I) as defined above is non-chiral in the final, deprotected oligonucleotide (i.e. when R¹ is hydrogen). The phosphorothioate internucleoside linkage of formula (I) will however be qualified in the present description as a non-chiral phosphorothioate internucleoside linkage even when R¹ is not hydrogen, as a precursor of a non-chiral linkage.

In the present description the term “alkyl”, alone or in combination, signifies a straight-chain or branched-chain alkyl group with 1 to 8 carbon atoms, particularly a straight or branched-chain alkyl group with 1 to 6 carbon atoms and more particularly a straight or branched-chain alkyl group with 1 to 3 carbon atoms. Examples of straight-chain and branched-chain C₁-C₈ alkyl groups are methyl, ethyl, propyl, isopropyl, butyl, isobutyl, tert.-butyl, the isomeric pentyls, the isomeric hexyls, the isomeric heptyls and the isomeric octyls, particularly methyl, ethyl, propyl, butyl and pentyl more particularly methyl, ethyl, propyl, isopropyl, isobutyl, tert.-butyl and isopentyl. Particular examples of alkyl are methyl, ethyl and propyl.

The term “alkoxy” or “alkyloxy”, alone or in combination, signifies a group of the formula alkyl-O— in which the term “alkyl” has the previously given significance, such as methoxy, ethoxy, n-propoxy, isopropoxy, n-butoxy, isobutoxy, sec.butoxy and tert.butoxy. Particular “alkoxy” are methoxy and ethoxy.

The terms “hydroxyl” and “hydroxy”, alone or in combination, signify the —OH group.

The terms “thiohydroxyl” and “thiohydroxy”, alone or in combination, signify the —SH group.

The term “amino”, alone or in combination, signifies the primary amino group (—NH₂), the secondary amino group (—NH—), or the tertiary amino group (—N—).

The term “sulfanyl”, alone or in combination, signifies the —S— group.

The term “azido”, alone or in combination, signifies the —N₃ group.

The term “oxy”, alone or in combination, signifies the —O— group.

The term “protecting group”, alone or in combination, signifies a group introduced into a molecule by chemical modification of a functional group to obtain chemoselectivity in a subsequent chemical reaction.

“Phosphate protecting group” is a protecting group of the phosphate group. Examples of phosphate protecting group are 2-cyanoethyl and methyl. A particular example of phosphate protecting group is 2-cyanoethyl.

“Hydroxyl protecting group” is a protecting group of the hydroxyl group and is also used to protect thiol groups. Examples of hydroxyl protecting groups are acetyl (Ac), benzoyl (Bz), benzyl (Bn), β-methoxyethoxymethyl ether (MEM), dimethoxytrityl (or bis-(4-methoxyphenyl)phenylmethyl) (DMT), trimethoxytrityl (or tris-(4-methoxyphenyl)phenylmethyl) (TMT), methoxymethyl ether (MOM), methoxytrityl [(4-methoxyphenyl)diphenylmethyl (MMT), p-methoxybenzyl ether (PMB), methylthiomethyl ether, pivaloyl (Piv), tetrahydropyranyl (THP), tetrahydrofuran (THF), trityl or triphenylmethyl (Tr), silyl ether (for example trimethylsilyl (TMS), tert-butyldimethylsilyl (TBDMS), tri-iso-propylsilyloxymethyl (TOM) and triisopropylsilyl (TIPS) ethers), methyl ethers and ethoxyethyl ethers (EE). Particular examples of hydroxyl protecting group are DMT and TMT, in particular DMT.

“Thiohydroxyl protecting group” is a protecting group of the thiohydroxyl group. Examples of thiohydroxyl protecting groups are those of the “hydroxyl protecting group”.

The term “nucleobase” refers to the base moiety of a nucleotide and covers both naturally occuring a well as non-naturally occurring variants. Thus, “nucleobase” covers not only the known purine and pyrimidine heterocycles but also heterocyclic analogues and tautomeres thereof. Examples of nucleobases include, but are not limited to adenine, guanine, cytosine, thymidine, uracil, xanthine, hypoxanthine, 5-methylcytosine, isocytosine, pseudoisocytosine, 5-bromouracil, 5-propynyluracil, 6-aminopurine, 2-aminopurine, inosine, diaminopurine and 2-chloro-6-aminopurine.

The term “nucleotide” as used herein, refers to a glycoside comprising a sugar moiety, a base moiety and a covalently linked group (linkage group), such as a phosphate or phosphorothioate internucleotide linkage group, and covers both naturally occurring nucleotides, such as DNA or RNA, and non-naturally occurring nucleotides comprising modified sugar and/or base moieties.

The term “glycoside” refers to a molecule in which a sugar is bound to another functional group via a glycosidic bond, i.e. a bond formed between the hemiacetal or hemiketal group of a saccharide (or a molecule derived from a saccharide) and the hydroxyl group of another molecule. The term “glycoside” also comprises compounds with bonds formed between hemiacetal (or hemiketal) groups of sugars and several chemical groups other than hydroxyls, such as —SR (thioglycosides), —SeR (selenoglycosides), —NR′R″ (N-glycosides) or —CR′R″R′″ (C-glycosides).

The term “solid support” refers to supports used for the solid phase synthesis, in particular of oligomeric compounds. Examples of solid phase support comprise crosslinked polystyrene (Primer Support 5G or NittoPhaseHL), controlled pore glass (CPG); oxalyl-controlled pore glass, silica-containing particles, such as porous glass beads and silica gel such as that formed by the reaction of trichloro-[3-(4-chloromethyl)phenyl]propylsilane and porous glass beads (PORASIL E®). Controlled pore glass is a particular useful solid phase support.

The term “oligonucleotide synthesis activator” refers to a compound capable of activating the reaction of an unprotected nucleoside with an incoming nucleoside phosphoramidite monomer. Examples of such oligonucleotide synthesis activators can be found in X. Wei, Tetrahedron 2013, 69, 3615-3637. Examples of oligonucleotide synthesis activators are azole based activators like 1H-tetrazole, 5-nitrophenyl-1H-tetrazole (NPT), 5-ethylthio-1H-tetrazole (ETT), 5-benzylthio-1H-tetrazole (BTT), 5-methylthio-1H-tetrazole (MTT), 5-mercapto-tetrazoles (MCT) and 4,5-dicyanoimidazole (DCI), or acidic salts like pyridinium hydrochloride, imidazoliuim triflate, benzimidazolium triflate, 5-nitrobenzimidazolium triflate, or weak acids such as 2,4-dinitrobenzoic acid or 2,4-dinitrophenol. 5-(3,5-bis(trifluoromethyl)phenyl)-1H-tetrazole is a particularly useful oligonucleotide synthesis activator.

The term “thiooxidation agent” refers to a reagent capable of converting a phosphoroamidite to a mercapto-phosphoroamidite. Examples of thiooxidation reagent are phenylacetyl disulfide, 3H-1,2-benzodithio1-3-one-1,1-dioxide, tetraethylthiuram disulfide (TETD), dibenzoyl tetrasulfide, bis(O,O-diisopropoxyphosphinothioyl) disulfide (S-Tetra), benzyltriethyl-ammonium tetrathiomolybate (BTTM), bis(p-toluenesulfonyl) disulfide, 3-ethoxy-1,2,4- dithiazoline-5-one (EDITH) and 1,2,4-dithiazolidine-3,5-dione (DtsNH) and in particular 3-amino-1,2,4-dithiazole-5-thione.

The term “capping” or “capping step” refers to the conversion of hydroxyl or thiohydroxyl groups that have not reacted during the oligonucleotide coupling (e.g. during the reaction of a compound of formula (V) with a compound of formula (VI) or during the reaction of a compound of formula (XI) with a compound of formula (XII)) into a protected hydroxyl or thiohydroxyl group. The capping thus hinders the reaction of said hydroxyl or thiohydroxyl groups in the next coupling steps. The capping step is for example conveniently performed by the reaction with acetic anhydride (Ac₂O) or phenoxyacetic anhydride (Pac-anhydride), for example in combination with activators like pyridine and N-methyl-imidazole, for example in THF or acetonitrile. The resulting protected hydroxyl or thiohydroxyl group is for example an acetate or thioacetate group.

The term “sugar modified nucleoside” refers to a nucleoside wherein the sugar is other than DNA or RNA.

The term “cation scavenger” designates a substance that reacts with, and thereby removes, free cations formed in a reaction. Examples of cation scavengers are silyl hydrides such as triethylsilane, triphenylsilane, triisopropylsilane, thiols like ethanedithiol, thiophenols like methoxy thiophenol, phenols and sulfides such as thioanisole. Particular cation scavengers are triethylsilane and methoxy thiophenol.

The invention thus relates in particular to:

A process of the invention, wherein the sulfur atom of the at least one non-chiral phosphorothioate internucleotide linkage of formula (I) or (II) is linked to the 3′ carbon atom or 5′ carbon atom of an adjacent nucleoside of the oligonucleotide;

A process of the invention wherein the oligonucleotide comprising at least one non-chiral phosphorothioate internucleoside linkage of formula (I) as defined above comprises a fragment of formula (III)

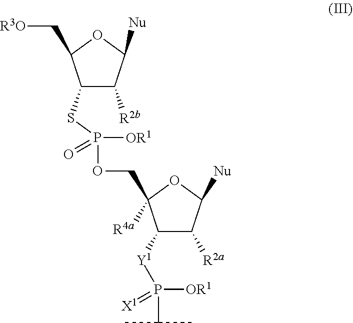

wherein
X¹ is oxygen or sulfur;
Y¹ is oxygen or sulfur;
provided that X¹ and Y¹ are not both sulfur at the same time;
each R¹ is independently as defined in claim 1;
   R^{2a} is hydrogen, hydroxyl, fluoro, alkyl, alkoxy, alkoxyalkoxy, —NH₂, alkylamino, dialkylamino, alkylcarbonylamino, azido, —SH, —CN, —CF₃, —OCF₃, alkylsulfanylalkoxy, aminooxyalkoxy, alkylaminooxyalkoxy, dialkylaminooxyalkoxy, aminocarbonylalkoxy, alkylaminocarbonylalkoxy or dialkylaminocarbonylalkoxy;
R^{4a} is hydrogen or hydroxyalkyl;
or R^{2a} and R^{4a} together form —CH₂O—, —CH₂NH—, —CH₂S—, —CH₂N(OR^{p})—, —CHCH₃O—, —C(CH₃)₂O—, —CH₂C(═CH₂)—, —CHCH₃C(═CH₂)—, —CHCH₃S—, —CH₂NR^{p}—, —CH₂CH₂O—, —CH₂CH₂CH₂O—, —CH₂OCH₂—, —CH(CH₂OCH₃)O—, —CH(CH₂CH₃)O— or —CH₂OCH₂O—;
provided that when Y¹ is sulfur, then R^{4a} is hydrogen;
R^{2b} ishydrogen, hydroxyl, fluoro, alkyl, alkoxy, alkoxyalkoxy, —NH₂, alkylamino, dialkylamino, alkylcarbonylamino, azido, —SH, —CN, —CF₃, —OCF₃, alkylsulfanylalkoxy, aminooxyalkoxy, alkylaminooxyalkoxy, dialkylaminooxyalkoxy, aminocarbonylalkoxy, alkylaminocarbonylalkoxy or dialkylaminocarbonylalkoxy;
R³ is a hydroxyl protecting group;
each R^{p} is independently alkyl; and
each Nu is independently a nucleobase;

A process of the invention, wherein the oligonucleotide comprising an internucleoside linkage of formula (II) as defined above comprises a fragment of formula (IV)

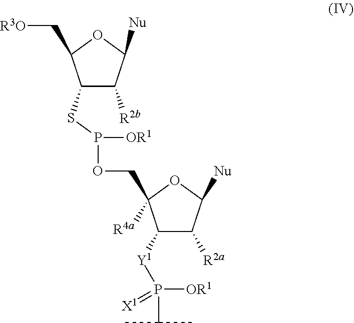

wherein X¹, Y¹, R¹, R^{2a}, R^{2b}, R³, R^{4a} and Nu are as defined above;

A process according to the invention wherein the oligonucleotide comprising at least one non-chiral phosphorothioate internucleoside linkage of formula (I) as defined above is reacted in the presence of acid to arrive at an oligonucleotide comprising a fragment of formula (V)

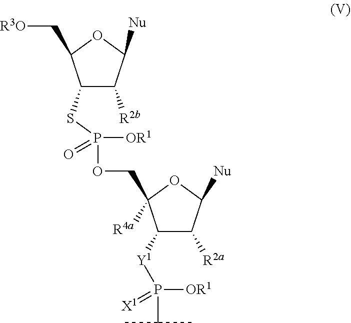

wherein X¹, Y¹, R¹, R^{2a}, R^{2b}, R^{4a} and Nu are as defined above;

A process of the invention wherein the oligonucleotide comprising a fragment of formula (V) as defined above is reacted in the presence of a compound of formula (VI)

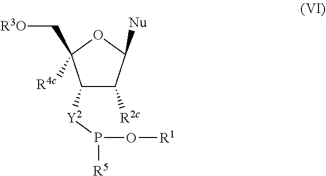

to arrive at an oligonucleotide comprising at least one non-chiral phosphorothioate internucleoside linkage of formula (I) as defined above comprising a fragment of formula (VII)

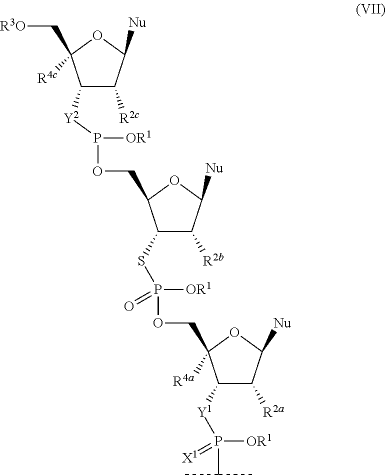

wherein
Y² is oxygen or sulfur;
R^{2c} is hydrogen, hydroxyl, fluoro, alkyl, alkoxy, alkoxyalkoxy, —NH₂, alkylamino, dialkylamino, alkylcarbonylamino, azido, —SH, —CN, —CF₃, —OCF₃, alkylsulfanylalkoxy, aminooxyalkoxy, alkylaminooxyalkoxy, dialkylaminooxyalkoxy, aminocarbonylalkoxy, alkylaminocarbonylalkoxy or dialkylaminocarbonylalkoxy;
R^{4c} is hydrogen or hydroxyalkyl;
or R^{2c} and R^{4c} together form —CH₂O—, —CH₂NH—, —CH₂S—, —CH₂N(OR^{p})—, —CHCH₃O—, —C(CH₃)₂O—, —CH₂C(═CH₂)—, —CHCH₃C(═CH₂)—, —CHCH₃S—, —CH₂NR^{p}—, —CH₂CH₂O—, —CH₂CH₂CH₂O—, —CH₂OCH₂—, —CH(CH₂OCH₃)O—, —CH(CH₂CH₃)O— or —CH₂OCH₂O—;
provided that when Y² is sulfur, then R^{4c} is hydrogen;
R⁵ is dialkylamino;
each R^{p} is independently alkyl; and
X¹, Y¹, R¹, R^{2a}, R^{2b}, R³, R^{4a} and Nu are as defined above;

A process according to the invention, wherein the oligonucleotide comprising a fragment of formula (VII) as defined above is reacted in the presence of a thiooxidation agent or iodine, wherein the concentration of iodine is between about 0.001 M and about 0.01 M, to arrive at an oligonucleotide comprising at least one non-chiral phosphorothioate internucleoside linkage of formula (I) as defined above comprising a fragment of formula (VIII)

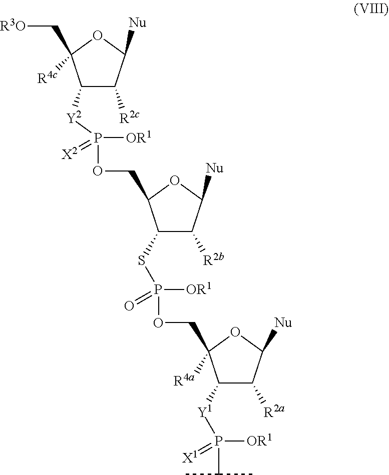

wherein
X² is oxygen or sulfur;
Y² is oxygen or sulfur;
provided that X² and Y² are not both sulfur at the same time; and
wherein X¹, Y¹, R¹, R^{2a}, R^{2b}, R^{2c}, R³, R^{4a}, R^{4c} and Nu are as defined above;

A process according to the invention wherein the oligonucleotide comprising a fragment of formula (VII) as defined above is reacted in the presence of a thiooxidation agent when Y² is oxygen;

A process according to of the invention wherein the oligonucleotide comprising a fragment of formula (VII) as defined above is reacted in the presence of iodine when Y² is sulfur;

A process according to the invention wherein the oligonucleotide comprising at least one non-chiral phosphorothioate internucleoside linkage of formula (I) as defined above comprises a fragment of formula (IX)

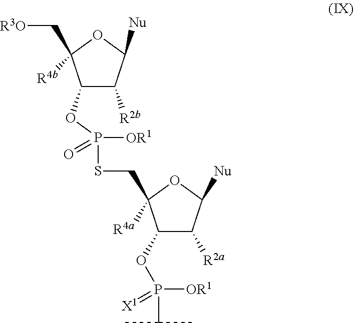

wherein
X¹ is oxygen or sulfur;
Y¹ is oxygen or sulfur;
each R¹ is independently as defined above;
R^{2a} is hydrogen, hydroxyl, fluoro, alkyl, alkoxy, alkoxyalkoxy, —NH₂, alkylamino, dialkylamino, alkylcarbonylamino, azido, —SH, —CN, —CF₃, —OCF₃, alkylsulfanylalkoxy, aminooxyalkoxy, alkylaminooxyalkoxy, dialkylaminooxyalkoxy, aminocarbonylalkoxy, alkylaminocarbonylalkoxy or dialkylaminocarbonylalkoxy;
R^{4a} is hydrogen or hydroxyalkyl; (hydroxymethyl);
or R^{2a} and R^{4a} together form —CH₂O—, —CH₂NH—, —CH₂S—, —CH₂N(OR^{p})—, —CHCH₃O—, —C(CH₃)₂O—, —CH₂C(═CH₂)—, —CHCH₃C(═CH₂)—, —CHCH₃S—, —CH₂NR^{p}—, —CH₂CH₂O—, —CH₂CH₂CH₂O—, —CH₂OCH₂—, —CH(CH₂OCH₃)O—, —CH(CH₂CH₃)O— or —CH₂OCH₂O—;
R^{2b} ishydrogen, hydroxyl, fluoro, alkyl, alkoxy, alkoxyalkoxy, —NH₂, alkylamino, dialkylamino, alkylcarbonylamino, azido, —SH, —CN, —CF₃, —OCF₃, alkylsulfanylalkoxy, aminooxyalkoxy, alkylaminooxyalkoxy, dialkylaminooxyalkoxy, aminocarbonylalkoxy, alkylaminocarbonylalkoxy or dialkylaminocarbonylalkoxy;
R^{4b} is hydrogen or hydroxyalkyl;
or R^{2b} and R^{4b} together form —CH₂O—, —CH₂NH—, —CH₂S—, —CH₂N(OR^{p})—, —CHCH₃O—, —C(CH₃)₂O—, —CH₂C(═CH₂)—, —CHCH₃C(═CH₂)—, —CHCH₃S—, —CH₂NR^{p}—, —CH₂CH₂O—, —CH₂CH₂CH₂O—, —CH₂OCH₂—, —CH(CH₂OCH₃)O—, —CH(CH₂CH₃)O— or —CH₂OCH₂O—;
R³ is a hydroxyl protecting group or a thiohydroxyl protecting group;
each R^{p} is independently alkyl; and
each Nu is independently a nucleobase;

A process according to the invention wherein the oligonucleotide comprising an internucleoside linkage of formula (II) as defined above comprises a fragment of formula (X)

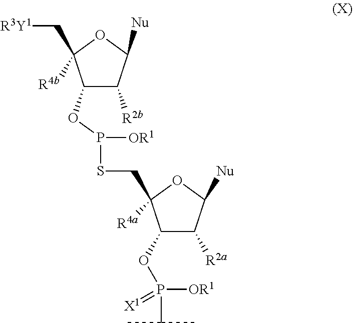

wherein X¹, Y¹, R¹, R^{2a}, R^{2b}, R³, R^{4a}, R^{4b} and Nu are as defined in the compound of formula (IX);

A process according to the invention wherein the oligonucleotide comprising at least one non-chiral phosphorothioate internucleoside linkage of formula (I) comprising a fragment of formula (IX) as defined above is reacted in the presence of acid to arrive at an oligonucleotide comprising a fragment of formula (XI)

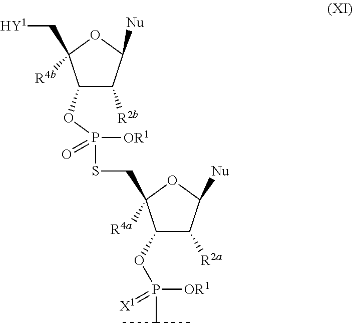

wherein X¹, Y¹, R¹, R^{2a}, R^{2b}, R³, R^{4a}, R^{4b} and Nu are as defined in the compound of formula (IX);

A process according to the invention wherein the oligonucleotide comprising a fragment of formula (XI) as defined above is reacted in the presence of a compound of formula (XII)

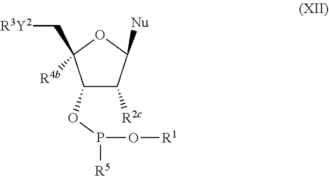

to arrive at an oligonucleotide comprising at least one non-chiral phosphorothioate internucleoside linkage of formula (I) according as defined above comprising a fragment of formula (XIII)

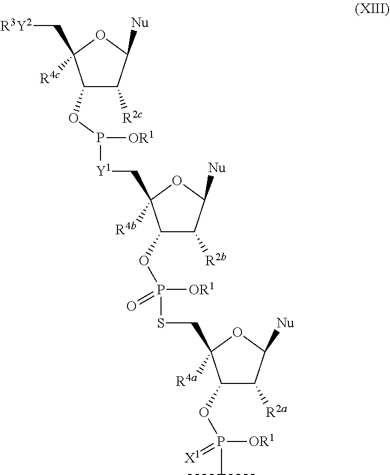

wherein
Y² is oxygen or sulfur;
R^{2c} is hydrogen, hydroxyl, fluoro, alkyl, alkoxy, alkoxyalkoxy, —NH₂, alkylamino, dialkylamino, alkylcarbonylamino, azido, —SH, —CN, —CF₃, —OCF₃, alkylsulfanylalkoxy, aminooxyalkoxy, alkylaminooxyalkoxy, dialkylaminooxyalkoxy, aminocarbonylalkoxy, alkylaminocarbonylalkoxy or dialkylaminocarbonylalkoxy;
R^{4c} is hydrogen or hydroxyalkyl;
or R^{2c} and R^{4c} together form —CH₂O—, —CH₂NH—, —CH₂S—, —CH₂N(OR^{p})—, —CHCH₃O—, —C(CH₃)₂O—, —CH₂C(═CH₂)—, —CHCH₃C(═CH₂)—, —CHCH₃S—, —CH₂NR^{p}—, —CH₂CH₂O—, —CH₂CH₂CH₂O—, —CH₂OCH₂—, —CH(CH₂OCH₃)O—, —CH(CH₂CH₃)O— or —CH₂OCH₂O—;
R³ is a hydroxyl protecting group or a thiohydroxyl protecting group;
R⁵ is dialkylamino;
each R^{p} is independently alkyl; and
wherein X¹, Y¹, R¹, R^{2a}, R^{2b}, R³, R^{4a}, R^{4b} and Nu are as defined in the compound of formula (IX);

A process according to the invention wherein the oligonucleotide comprising a fragment of formula (XIII) as defined above is reacted in the presence of a thiooxidation agent or iodine, wherein the concentration of iodine is between about 0.001 M and about 0.01 M, to arrive at an oligonucleotide comprising at least one non-chiral phosphorothioate internucleoside linkage of formula (I) as defined above comprising a fragment of formula (XIV)

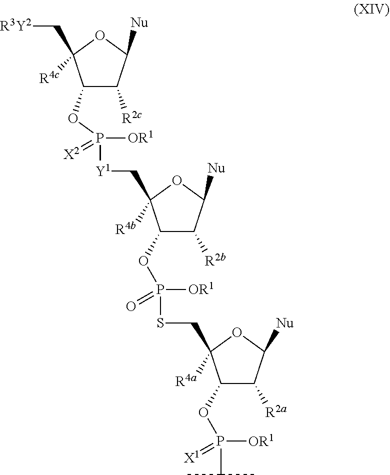

wherein
Y¹ is oxygen or sulfur;
X² is oxygen or sulfur;
provided that Y¹ and X² are not both sulfur at the same time; and
wherein X¹, Y², R¹, R^{2a}, R^{2b}, R^{2c}, R³, R^{4a}, R^{4b} and Nu are as defined in the compound of formula (XIII);

A process according to the invention wherein the oligonucleotide comprising a fragment of formula (XIII) as defined above is reacted in the presence of a thiooxidation agent when Y¹ is oxygen;

A process according to the invention wherein the oligonucleotide comprising a fragment of formula (XIII) as defined above is reacted in the presence of iodine when Y¹ is sulfur;

A process according to the invention wherein the oligonucleotide comprising at least one non-chiral phosphorothioate internucleoside linkage of formula (I) as defined above comprises 1 to 8 internucleoside linkages of formula (I), preferably 1 to 6 internucleoside linkages of formula (I);

A process according to the invention wherein the concentration of iodine is between about 0.001 M and about 0.005 M, preferably between about 0.002 M and about 0.005 M;

A process according to the invention wherein R¹ is cyanoethyl;

A process according to the invention wherein the hydroxyl protecting group or the thiohydroxyl protecting group is bis-(4-methoxy-phenyl)-phenyl-methyl;

A process according to the invention wherein R⁵ is diisopropylamino;

A process according to the invention wherein each Nu is independently selected from adenine, thymine, uracil, guanine and cytosine;

A process according to the invention wherein the oligonucleotide comprising at least one non-chiral phosphorothioate internucleoside linkage of formula (I) as defined above is bound to a solid support for solid phase synthesis;

A process according to the invention wherein the acid is dichloroacetic acid or trichloroacetic acid;

A process according to the invention wherein the oligonucleotide comprising a fragment of formula (V) as defined above is reacted in the presence of a compound of formula (VI) as defined above in the presence of an oligonucleotide synthesis activator;

A process according to the invention wherein the oligonucleotide comprising a fragment of formula (XI) as defined above is reacted in the presence of a compound of formula (XII) as defined above in the presence of an oligonucleotide synthesis activator;

A process according to the invention wherein the oligonucleotide synthesis activator is 5-(3,5-bis(trifluoromethyl)phenyl)-1H-tetrazole;

A process according to the invetion wherein the thiooxidation agent is 3-amino-1,2,4-dithiazole-5-thione;

A process according to the invention wherein the step of reacting an oligonucleotide comprising an internucleoside linkage of formula (II) as defined above in the presence of iodine is followed by a capping step;

A process according to the invention wherein the phosphate protecting group R¹ of the oligonucleotide comprising at least one non-chiral phosphorothioate internucleoside linkage of formula (I) as defined above is removed to arrive at an oligonucleotide comprising at least one non-chiral phosphorothioate internucleoside linkage of formula (XV)

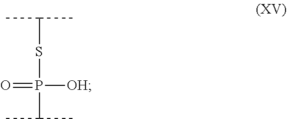

A process according to the invention wherein the the phosphate protecting group R¹ is removed by the reaction of the oligonucleotide comprising at least one non-chiral phosphorothioate internucleoside linkage of formula (I) as defined above in the presence of ammonia, ammonium hydroxide or a mixture of ammonium hydroxide and methylamine;

A process according to the invention wherein the the phosphate protecting group R¹ is removed by the reaction of the oligonucleotide comprising at least one non-chiral phosphorothioate internucleoside linkage of formula (I) as defined above in the presence of aqueous ammonia;

A process according to the invention wherein the the oligonucleotide comprising at least one non-chiral phosphorothioate internucleoside linkage of formula (I) as defined above is cleaved from the solid support to which it is bound;

A process according to the invention wherein the the oligonucleotide comprising at least one non-chiral phosphorothioate internucleoside linkage of formula (I) as defined above is cleaved from the solid support to which it is bound by reaction in the presence of aqueous ammonia;

A process according to the invention wherein the oligonucleotide comprising at least one non-chiral phosphorothioate internucleoside linkage of formula (I) as defined above is reacted in the presence of acid to remove hydroxyl protecting groups or thiohydroxyl protecting groups;

An oligonucleotide manufactured according to a process of the invention;

An oligounucleotide comprising at least one non-chiral phosphorothioate internucleoside linkage of formula (I) as defined above comprising 7 to 31 nucleotides;

An oligounucleotide comprising at least one non-chiral phosphorothioate internucleoside linkage of formula (I) as defined above wherein the sulfur atom of the at least one phosphorothioate internucleotide linkage of formula (I) is linked to the 3′ carbon atom or 5′ carbon atom of an adjacent nucleoside of the oligonucleotide;

An oligounucleotide comprising at least one non-chiral phosphorothioate internucleoside linkage of formula (I) as defined above wherein the oligonucleotide comprises at least one nucleotide of formula (XVI)

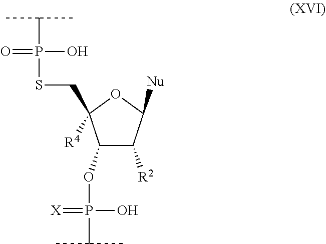

wherein
X is oxygen or sulfur;
R² and R⁴ together form —CH₂O—, —CH₂NH—, —CH₂S—, —CH₂N(OR^{p})—, —CHCH₃O—, —C(CH₃)₂O—, —CH₂C(═CH₂)—, —CHCH₃C(═CH₂)—, —CHCH₃S—, —CH₂NR^{p}—, —CH₂CH₂O—, —CH₂CH₂CH₂O—, —CH₂—O—CH₂—, —CH(CH₂OCH₃)O—, —CH(CH₂CH₃)O— or —CH₂OCH₂O—;
each R^{p} is independently alkyl; and
Nu is a nucleobase;

An oligounucleotide comprising at least one non-chiral phosphorothioate internucleoside linkage of formula (I) as defined above wherein the oligonucleotide comprises at least one nucleotide of formula (XVI)

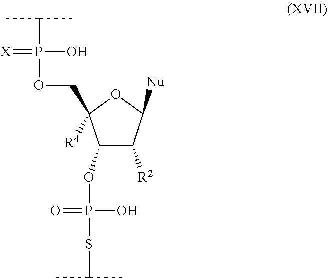

wherein
X is oxygen or sulfur;
R² and R⁴ together form —CH₂O—, —CH₂NH—, —CH₂S—, —CH₂N(OR^{p})—, —CHCH₃O—, —C(CH₃)₂O—, —CH₂C(═CH₂)—, —CHCH₃C(═CH₂)—, —CHCH₃S—, —CH₂NR^{p}—, —CH₂CH₂O—, —CH₂CH₂CH₂O—, —CH₂—O—CH₂—, —CH(CH₂OCH₃)O—, —CH(CH₂CH₃)O— or —CH₂OCH₂O—;
each R^{p} is independently alkyl; and
Nu is a nucleobase;

An oligounucleotide according to the invention comprising at least two internucleoside linkages of formula (I) as defined above;

An oligounucleotide according to the invention comprising at least two or at least three internucleoside linkages of formula (I) as defined above;

An oligonucleotide according to the invention comprising at least one LNA nucleoside;

An oligonucleotide according to the invention comprising at least one sugar modified nucleoside;

An oligonucleotide according to the invention wherein the at least one sugar modified nucleoside is independently selected from affinity enhancing 2′ sugar modified nucleosides;

An oligonucleotide according to the invention, wherein the at least one sugar modified nucleoside is independently selected from 2′-alkoxy-RNA, in particular 2′-methoxy-RNA, 2′-alkoxyalkoxy-RNA, in particular 2′-methoxyethoxy-RNA, 2′-amino-DNA, 2′-fluoro-RNA, 2′-fluoro-ANA nucleoside and LNA nucleosides.

A process or oligonucleotide of the invention wherein R^{2a} is hydrogen, hydroxyl, fluoro, alkoxy or alkoxyalkoxy;

A process or oligonucleotide of the invention wherein R^{2a} is hydrogen, hydroxyl, fluoro, methoxy or methoxyethoxy;

A process or oligonucleotide of the invention wherein R^{4a} is hydrogen or hydroxyalkyl;

A process or oligonucleotide of the invention wherein R^{4a} is hydrogen or hydroxymethyl;

A process or oligonucleotide of the invention wherein R^{4a} is hydrogen;

A process or oligonucleotide of the invention wherein R^{2b} is hydrogen, hydroxyl, fluoro, alkoxy or alkoxyalkoxy;

A process or oligonucleotide of the invention wherein R^{2b} is hydrogen, hydroxyl, fluoro, methoxy or methoxyethoxy;

A process or oligonucleotide of the invention wherein R^{4b} is hydrogen or hydroxyalkyl;

A process or oligonucleotide of the invention wherein R^{4b} is hydrogen or hydroxymethyl;

A process or oligonucleotide of the invention wherein R^{4b} is hydrogen;

A process or oligonucleotide of the invention wherein R^{2c} is hydrogen, hydroxyl, fluoro, alkoxy or alkoxyalkoxy;

A process or oligonucleotide of the invention wherein R^{2c} is hydrogen, hydroxyl, fluoro, methoxy or methoxyethoxy;

A process or oligonucleotide of the invention wherein R^{4c} is hydrogen or hydroxyalkyl;

A process or oligonucleotide of the invention wherein R^{4c} is hydrogen or hydroxymethyl;

A process or oligonucleotide of the invention wherein R^{4c} is hydrogen;

A process or oligonucleotide of the invention wherein R^{2a} and R^{4a} together form —CH₂O—;

A process or oligonucleotide of the invention wherein R^{2b} and R^{4b} together form —CH₂O—;

A process or oligonucleotide of the invention wherein R^{2c} and R^{4c} together form —CH₂O—;

A process or oligonucleotide of the invention wherein each R^{p} is independently methyl, ethyl or propyl;

An oligonucleotide of the invention wherein R² and R⁴ together form —CH₂O—;

A process or oligonucleotide of the invention wherein the oligonucleotide comprises between 1 and 8 non-chiral phosphorothioate internucleoside linkage of formula (I) as defined above; and

A pharmaceutical composition comprising an oligonucleotide according to the invention.

The invention also relates to a process according to the invention wherein the oligonucleotide comprising at least one non-chiral phosphorothioate internucleoside linkage of formula (I), comprising a fragment of formula (IX) as defined above wherein Y¹ is sulfur, is reacted in the presence of acid and of a cation scavenger to arrive at an oligonucleotide comprising a fragment of formula (XI).

The invention thus relates in particular to the DMT deprotection of a DMT-protected 5′-S DNA or 5′-S LNA monomer or of a DMT-protected terminal 5′-S DNA or 5′S-LNA nuceloside in an oligonucleotide, with an acid and a cation scavenger.

The acid is for example trichloroacetic acid or trifluoroacetic acid.

Examples of cation scavengers are triethylsilane, methoxy thiophenol and mixtures thereof.

Examples of advantageous combinations of acid and cation scavengers are trichloroacetic acid or trifluoroacetic acid with triethylsilane and/or methoxy thiophenol.

The acid, in particular trichloroacetic acid, is advantageously used at 5-10% (w/v).The acid, in particular trifluororoacetic acid, is advantageously used at 1-10% (v/v), in particular at 1-5% (v/v).

The cation scavenger is advantageously used at 2-30% (v/v).

The cation scavenger triethylsilane is advantageously used at 5-30% (v/v).

The cation scavenger p-methoxy thiophenol is advantageously used at 2-10% (v/v).

The DMT deprotection according to the invention is advantageously done in dichloromethane.

Advantageous DMT deprotection according to the invention are therefore:
6 applications of 200 μL for 45 sec of 10% (w/v) trichloroacetic acid in the presence of 20% (v/v) triethylsilane in CH₂Cl₂;
6 applications of 200 μL for 45 sec of 10% (w/v) trichloroacetic acid in the presence of 5% (v/v) p-Methoxy thiophenol in CH₂Cl₂;
6 applications of 200 μL for 45 sec of 5% (v/v) trifluoroacetic acid in the presence of 20% (v/v) triethylsilane in CH₂Cl₂;
3 applications of 200 μL for 45 sec of 5% (v/v) trifluoroacetic acid in the presence of 5% (v/v) p-Methoxy thiophenol and 20% (v/v) of triethylsilane in CH₂Cl₂;
3-6 applications of 200 μL for 45 sec of 5-10% (w/v) trichloroacetic acid in the presence of 5-30% (v/v) triethylsilane in CH₂Cl₂ and/or 2-10% of p-Methoxy thiophenol in CH₂Cl₂; or
3-6 applications of 200 μL for 45 sec of 1-10% (v/v) trifluoroacetic acid in the presence of 5-30% (v/v) triethylsilane in CH₂Cl₂ and/or 2-10% of p-Methoxy thiophenol in CH₂Cl₂.

The synthesis of oligonucleotide phosphorothioates containing one or more 2′,3′-dideoxy-3′-mercapto nucleotides can for example be performed by solid phase oligonucleotide synthesis using controlled pore glass (CPG) modified with an universal linker as the support. To this support the first nucleotide (either DNA or LNA) can be coupled as the corresponding phosphoramidite using a tetrazole derivative as the acidic activator. Coupling conditions include the use of 10 equivalents of phosphoramidite as well as triple couplings in order to ensure complete reactions. The resulting phosphite intermediate can then subjected to a thiooxidation resulting in a phosphorothioate linkage. After capping of potential unreacted 5′-hydroxy groups by acetylation, the protected mononucleotide thus immobilized on the solid support is then deprotected by an acid promoted cleavage of the dimethoxytrityl protecting group on the 5′-hydroxy group on the ribose sugar. Sequential repetition of this synthesis cycle using appropriate phosphoramidite building blocks allows for the build-up of the desired oligonucleotide sequence. Due to their lower coupling efficiency, 2′,3′-dideoxy-3′-mercapto nucleotides are preferably coupled 10 times using 4 equivalents of the corresponding phosphoramidite and a coupling time of 15 min for each coupling. The resulting thiophosphite intermediate can then be oxidized to the corresponding thiophosphate using molecular iodine at a concentration below 2 mM. After a capping step, a standard acid promoted removal of the dimethoxytrityl protecting group as described above finishes the synthetic cycle.

2′,5′-dideoxy-5′-mercapto nucleotides are introduced using triple couplings and 10 equivalents of phosphoramidite in the presence of a tetrazole derivative as the acidic activator. The resulting phosphite intermediates are either oxidized to the corresponding phosphates using iodine or subjected to thiooxidation resulting in a phosphorothioate linkage followed by a capping step. In order to liberate the 5′-thiol from the dimethoxytrityl protecting group an extended acid treatment is necessary. The free 5′-thiol thus obtained can then undergo coupling to the subsequent nucleotide resulting in a thiophosphite intermediate. This intermediate is then oxidized to the corresponding thiophosphate using molecular iodine at a concentration below 2 mM. Capping and detritylation finishes the synthetic cycle.

Once the desired oligonucleotide sequence has been built up by repetitions of the synthesis cycle, it can be cleaved off the solid support and globally deprotected by the treatment with aqueous ammonia.

These steps are schematically described in the following schemes which have no limiting character. X¹, Y¹, R¹, R^{2a}, R^{2b}, R³, R^{4a}, R^{4b} and Nu are as defined above.

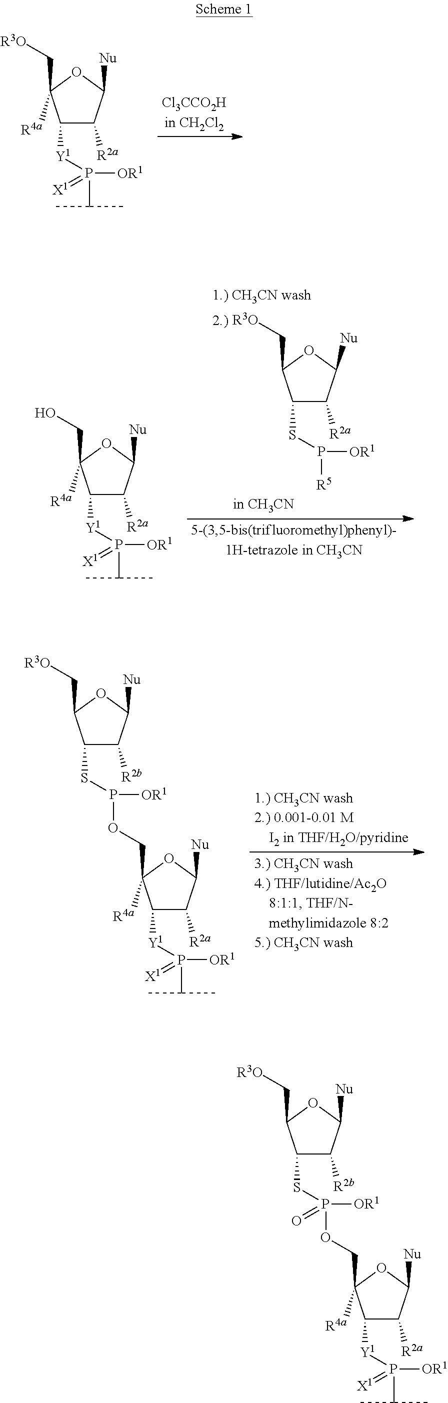

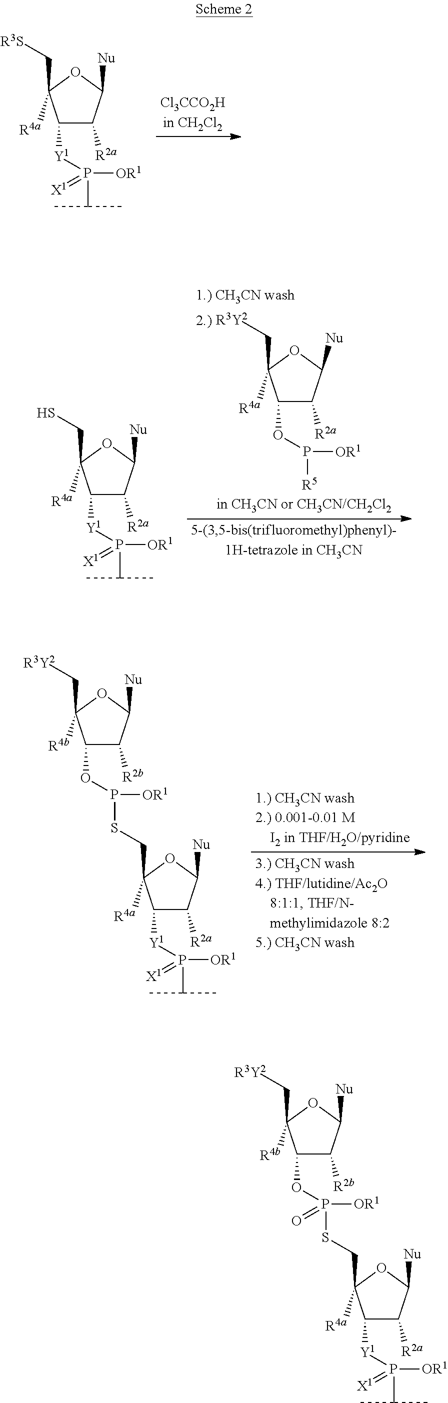

5′S-LNA monomers (compounds of formula (XII) wherein Y²═S and R^{2c} and R^{4c} together form —CH₂O—, —CH₂NH—, —CH₂S—, —CH₂N(OR^{p})—, —CHCH₃O—, —C(CH₃)₂O—, —CH₂C(═CH₂)—, —CHCH₃C(═CH₂)—, —CHCH₃S—, —CH₂NR^{p}—, —CH₂CH₂O—, —CH₂CH₂CH₂O—, —CH₂OCH₂—, —CH(CH₂OCH₃)O—, —CH(CH₂CH₃)O— or —CH₂OCH₂O—) can be synthesized according to the procedure depicted in scheme 3. R³ is as defined above.

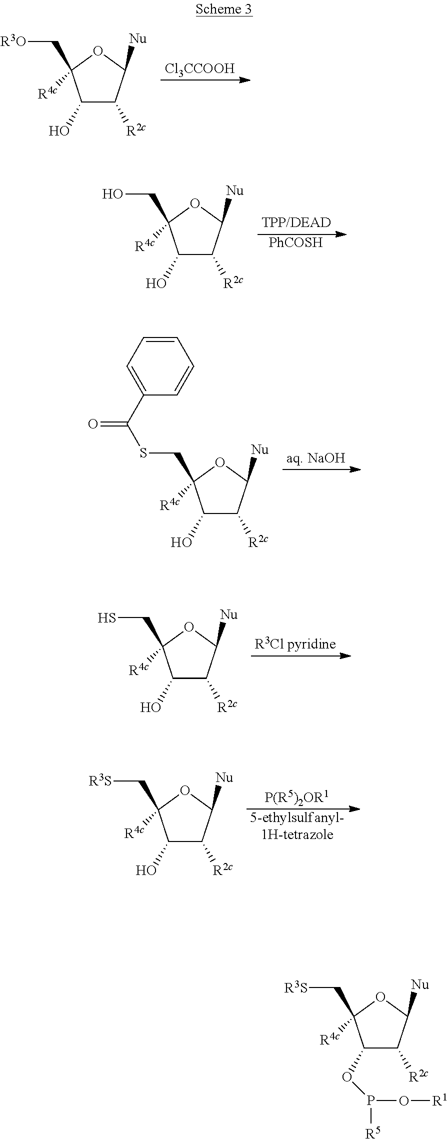

Starting from protected nucleosides the 5′-hydroxy group is liberated by acid treatment. The 5′-sulfur atom is introduced by means of a Mitsunobu reaction using thiobenzoic acid as the nucleophile followed by hydrolysis of the resulting thioester. After protection of the free thiol the desired phosphoramidite building block is obtained by phosphitylation of the 3′-hydroxy group with a suitable phosphordiamidite in the presence of a tetrazole derivative as an acidic activator.

The invention will now be ilustrated by the following examples which have no limiting character.

### EXAMPLES

### Example 1

### Monomer synthesis 5′S-LNA monomer Synthesis

To a solution of Cl₃CCOOH (2.98 g, 18.23 mmol) in CH₂Cl₂ (150 ml) was added N-[9-(1-{[bis(4-methoxyphenyl)(phenyl)methoxy]methyl}-7-hydroxy-2,5-dioxabicyclo[2.2.1]heptan-3-yl)-9H-purin-6-yl]benzamide (10 g, 14.58 mmol) at 25° C. Then the reaction mixture was stirred for 3 h at 25° C. Volatiles were removed under reduced pressure and the resulting crude was purified by combiflash (10% MeOH in CH₂Cl₂) to get N-{9-[7-hydroxy-1-(hydroxymethyl)-2,5-dioxabicyclo[2.2.1]heptan-3-yl]-9H-purin-6-yl}benzamide (5 g, 89%) as white solid. (MS: (ESI): m/z=383.8 [M+H]⁺).

To an ice cooled solution of triphenyl phosphine (10.26 g, 39.13 mmol) in anhydrous THF (150.0 mL) was added diethyl azodicarboxylate (6.14 mL, 39.13 mmol) and the reaction mixture was stirred at 0° C. for 30 min. PhCOSH (4.62 mL, 39.13 mmol) was added drop wise to the reaction mixture and it was stirred at 0° C. for another 30 min. N-{9-[7-hydroxy-1-(hydroxymethyl)-2,5-dioxabicyclo[2.2.1]heptan-3-yl]-9H-purin-6-yl}benzamide (5.0 g, 13.04 mmol) was added and the reaction mixture was stirred at 0° C. for 2 h followed by stiffing at room temperature for 2 h. The reaction mixture was diluted with water (200 mL) and extracted with ethyl acetate (120 mL×3). The combined organic layers were washed with NaHCO₃ (100 mL), dried over Na₂SO₄, filtered and evaporated under reduced pressure to get N-(9-{1-[(benzoylsulfanyl)methyl]-7-hydroxy-2,5-dioxabicyclo[2.2.1]heptan-3-yl}-9H-purin-6-yl)benzamide (25 g, crude) as a yellow viscous oil. (MS: (ESI): m/z=504.3 [M+H]⁺).

An aqueous solution of NaOH (0.5 M, 238 mL) as well as a mixture of THF-MeOH (6:4, 250 mL) were bubbled with argon for 30 min. N-(9-{1-[(benzoylsulfanyl)methyl]-7-hydroxy-2,5-dioxabicyclo[2.2.1]heptan-3-yl}-9H-purin-6-yl)benzamide (20 g, crude) was dissolved in the argon purged solution of THF-MeOH (6:4, 250 mL) under argon and cooled to 0° C. to −5° C. To this solution was added the NaOH solution (0.5 M, 238 mL, 119.16 mmol) and the reaction mixture was stirred at 0° to −5° C. for 30 min. A solution of citric acid (30.04 g, 142.98 mmol) was added at 0° C. The reaction mixture was diluted with a saturated NaHCO₃ solution (300 mL) and extracted with ethyl acetate (200 mL×3). The combined organic layers were washed with brine, dried over sodium sulfate, filtered and evaporated under reduced pressure to get N-{9-[7-hydroxy-1-(sulfanylmethyl)-2,5-dioxabicyclo[2.2.1]heptan-3-yl]-9H-purin-6-yl}benzamide (20 g, crude) as an off white viscous oil. (MS: (ESI): m/z=400.2 [M+H]⁺).

To a solution of N-{9-[7-hydroxy-1-(sulfanylmethyl)-2,5-dioxabicyclo[2.2.1]heptan-3-yl]-9H-purin-6-yl}benzamide (20 g, crude) in anhydrous pyridine (20 mL, argon purged) was added DMTrCl (5.09 g, 15.02 mmol) at 25° C. and the reaction mixture was stirred at 25° C. for 4 h. Volatiles were removed under reduced pressure and the reaction mixture was diluted with CH₂Cl₂ (300 mL). The organic layer was washed with a sat. NaHCO₃ solution (100 mL×2) followed by brine (100 mL), dried over Na₂SO₄, filtered and evaporated under reduced pressure. The resulting crude material was purified by combiflash (2% MeOH in CH₂Cl₂ containing 0.5% triethylamine) to get N-{9-[1-({[bis(4-methoxyphenyl) (phenyl)methyl]sulfanyl}methyl)-7-hydroxy-2,5-dioxabicyclo[2.2.1]heptan-3-yl]-9H-purin-6-yl}benzamide (5 g, 68% over 3 steps) as a pale yellow solid. (MS: (ESI): m/z=702.14 [M+H]⁺).

A solution of 5-ethylmercapto-1H-tetrazole (1.3 g, 9.97 mmol, 0.25 M solution in 38.4 mL dry acetonitrile) was added to a stirred solution of N-{9-[1-({[bis(4-methoxyphenyl)(phenyl)methyl]sulfanyl}methyl)-7-hydroxy-2,5-dioxabicyclo[2.2.1]heptan-3-yl]-9H-purin-6-yl}benzamide (3.5 g, 4.99 mmol) in dry CH₂Cl₂ (120 mL) under argon at room temperature followed by the addition of 2-cyanoethyl tetraisopropylphosphorodiamodite (3.17 mL, 9.98 mmol). After stiffing at room temperature for 4 h the reaction mixture was diluted with CH₂Cl₂ (300 mL) and poured into a sat. NaHCO₃ solution (100 mL). The organic layer was separated off and the aqueous layer was extracted with CH₂Cl₂ (70 mL×2). The combined organic layers were dried over Na₂SO₄, filtered and evaporated under reduced pressure. The resulting crude compound was purified by combiflash (10-20% ACN in DCM) to get (2.7 g) impure. Using the same protocol two further batches were performed at a 1.0 g and a 2.5 g scale resulting in 0.6 g and 2.5 g of impure product respectively. The impure compound thus obtained from different batches was mixed and repurified to get N-{9-[1-({[bis(4-methoxyphenyl)(phenyl)methyl]sulfanyl}methyl)-7-({[bis(propan-2-yl)amino](2-cyanoethoxy)phosphanyl}oxy)-2,5-dioxabicyclo[2.2.1]heptan-3-yl]-9H-purin-6-yl}benzamide (5′-Mercapto-LNA adenosine phosphoramidite, 4.0 g, 44%) as a white solid. (MS: (ESI): m/z=901.6 [M+H]⁺).

### Example 2

### 5′-S and 3′-S DNA monomer Synthesis

### 3′S DNA Phosphoramidite

To an ice-cooled solution of N-{9-[(2R,4S,5R)-5-{[bis(4-methoxyphenyl)(phenyl)methoxy]methyl}-4-hydroxyoxolan-2-yl]-9H-purin-6-yl}benzamide) (25 g, 38.01 mmol) and 4-nitrobenzoic acid (12.70 g, 76.02 mmol) in dry THF (2.0 L) was added triphenyl phosphine (39.89 g, 152.04 mmol) followed by the drop wise addition of diisopropyl azodicarboxylate (30.74 g, 152.04 mmol) and the reaction mixture was stirred at 0° C. for 2 h. Progress of the reaction was monitored by TLC. Volatiles were removed under reduced pressure to get a pale brown thick mass crude compound which was purifiedby combiflash (70-90% EtOAc in hexane) to afford N-{9-[(2R,4S,5R)-5-{[bis(4-methoxyphenyl)(phenyl)methoxy]methyl}-4-hydroxyoxolan-2-yl]-9H-purin-6-yl}benzamide (40 g, crude) as an off-white solid. (MS: (ESI): m/z=807.6 [M+H]⁺).

To an ice-cooled solution of N-{9-[(2R,4S,5R)-5-{[bis(4-methoxyphenyl)(phenyl)methoxy]methyl}-4-hydroxyoxolan-2-yl]-9H-purin-6-yl}benzamide (40 g, crude) in dry methanol (500 mL) was added K₂CO₃ (6.85 g, 49.58 mmol) under an argon atmosphere and the reaction mixture was stirred at 0° C. for 2 h. Volatiles were removed under reduced pressure to get crude compound which was dissolved in ethyl acetate (300 mL) and washed with water (100 mL). The aqueous layer was further extracted with EtOAc (2×100 mL). The combined organic layers were dried over Na₂SO₄ and concentrated under reduced pressure to get crude compound which was purified by combiflash (5% MeOH in EtOAc) to afford N-{9-[(2R,4R,5R)-5-{[bis(4-methoxyphenyl)(phenyl) methoxy]methyl}-4-hydroxyoxolan-2-yl]-9H-purin-6-yl}benzamide (13 g, 52% after two steps) as an off-white solid. (MS: (ESI): m/z=658.4 [M+H]⁺).

To an ice-cooled solution of triphenyl phosphine (15.55 g, 59.30 mmol) in dry THF (350 mL) was added diethyl azodicarboxylate (9.30 mL, 59.30 mmol) drop wise and the reaction mixture was stirred at 0° C. under an argon atmosphere for 30 min. Thiobenzoic acid (7.00 mL, 59.30 mmol) was added and the resulting mixture was stirred at 0° C. for 30 min. After the addition of N-{9-[(2R,4R,5R)-5-{[bis(4-methoxyphenyl)(phenyl)methoxy]methyl}-4-hydroxyoxolan-2-yl]-9H-purin-6-yl}benzamide (13.0 g, 19.77 mmol) in dry THF (150 mL) stirring was continued at 0° C. for 3 h, allowed to warm to room temperature and stirred at 25° C. for 16 h. Volatiles were removed under reduced pressure to get crude compound which was purified by combiflash (50% ethyl acetate in hexane containing 0.5% triethylamine) to afford N-{9-[(2R,4S,5R)-4-(benzoylsulfanyl)-5-{[bis(4-methoxyphenyl)(phenyl)methoxy]methyl}oxolan-2-yl]-9H-purin-6-yl}benzamide (8.1g, 53%) as yellow solid. (MS: (ESI): m/z=777.6 [M+H]⁺).

An aqueous solution of NaOH (0.5 M, 38.5 mL) as well as a mixture of THF-MeOH (6:4, 75 mL) were bubbled with argon for 30 min. N-{9-[(2R,4S,5R)-4-(benzoylsulfanyl)-5-{[bis (4-methoxyphenyl)(phenyl) methoxy]methyl}oxolan-2-yl]-9H-purin-6-yl}benzamide (5.0 g, 6.43 mmol) was added to the solution of MeOH/THF (75.0 mL) under argon and cooled to 0 to −5° C. To this solution was added the above NaOH solution (38.5 mL, 19.28 mmol) and the reaction mixture was stirred at 0 to −5° C. for 30 min. After neutralization with citric acid at 0° C., a saturated NaHCO₃ solution was added to the reaction mixture and the solution was extracted with ethyl acetate (200 mL×3). Combined organic layers were washed with brine, dried over Na₂SO₄ and concentrated under reduced pressure to get N-{9-[(2R,4S,5R)-5-{[bis(4-methoxyphenyl)(phenyl)methoxy]methyl}-4-sulfanyloxolan-2-yl]-9H-purin-6-yl}benzamide (5.3 g, crude) as pale yellow solid. (MS: (ESI): m/z=673.6 [M+H]⁺).

A solution of 5-ethylmercapto-1H-tetrazole (1.64 g, 12.62 mmol, 0.25 M solution in 50.0 mL dry acetonitrile) was added to a stirred solution of N-{9-[(2R,4S,5R)-5-{[bis(4-methoxyphenyl)(phenyl)methoxy]methyl}-4-sulfanyloxolan-2-yl]-9H-purin-6-yl}benzamide (8.5 g, crude) in dry CH₂Cl₂ (80.0 mL) under an argon atmosphere at room temperature followed by the addition of 2-cyanoethyl tetra isopropyl phosphorodiamidite (6.004 mL, 18.92 mmol) and the reaction mixture was stirred at 25° C. for 1 h. Then the reaction mixture was diluted with CH₂Cl₂ (300 mL) and poured into a saturated NaHCO₃ solution (200 mL). The organic layer was separated off and the aqueous layer was extracted with CH₂Cl₂ (100 mL×2). The combined organic layers were dried over sodium sulfate and concentrated under reduced pressure to get crude compound which was purified by combiflash (50% acetonitrile in CH₂Cl₂) to get N-{9-[(2R,4S,5R)-5-{[bis(4-methoxyphenyl)(phenyl)methoxy]methyl}-4-({[bis(propan-2-yl)amino](2-cyanoethoxy)phosphanyl}sulfanyl)oxolan-2-yl]-9H-purin-6-yl}benzamide (3′-Mercapto-DNA adenosine phosphoramidite, 6.3 g, 70% over 2 steps) as an off-white solid. (MS: (ESI): m/z=874.7 [M+H]⁺).

### 5′S DNA Phosphoramidite

To an ice cooled solution of N-{9-[(2R,4S,5R)-5-{[bis(4-methoxyphenyl)(phenyl)methoxy]methyl}-4-hydroxyoxolan-2-yl]-9H-purin-6-yl}benzamide (20 g, 30.41 mmol) in CHCl₃ (300 mL) was added a solution of Cl₃CCOOH (2.48 g, 15.20 mmol) in CHCl₃ (50 ml). Then the reaction mixture was stirred for 2 h at 0° C. Triethylamine (8.5 mL, 60.82 mmol) was added and volatiles were removed under reduced pressure. The resulting crude compound was purified by flash column chromatography (10% MeOH in CH₂Cl₂). The solid thus obtained was washed with water and dried to get N-{9-[(2R,4S,5R)-4-hydroxy-5-(hydroxymethyl)oxolan-2-yl]-9H-purin-6-yl}benzamide (7 g, 65%) as white solid. (MS: (ESI): m/z=356.0 [M+H]⁺).

To an ice cooled solution of triphenyl phosphine (10.33 g, 39.40 mmol) in anhydrous THF (200 mL) was added diethyl azodicarboxylate (6.18 mL, 39.40 mmol) and the reaction mixture was stirred at 0° C. for 30 min. PhCOSH (4.65 mL, 39.40 mmol) was added drop wise and stirring was continued at 0° C. for another 30 min. To the resulting reaction mixture was added N-{9-[(2R,4S,5R)-4-hydroxy-5-(hydroxymethyl)oxolan-2-yl]-9H-purin-6-yl}benzamide (7 g, 19.70 mmol). After stirring at 0° C. for 2 h, the mixture was allowed to warm to room temperature and stirred at 25° C. for 2 h. Volatiles were removed under reduced pressure and the crude compound thus obtained was purified by combiflash (2% MeOH in CH₂Cl₂) to get N-{9-[(2R,4S,5S)-5-[(benzoylsulfanyl)methyl]-4-hydroxyoxolan-2-yl]-9H-purin-6-yl}benzamide (5 g, 53%) as pale yellow viscous oil. (MS: (ESI): m/z=475.6 [M+H]⁺).

An aqueous solution of NaOH (0.5 M, 25.25 mL) as well as a mixture of THF-MeOH (6:4, 75 mL) were bubbled with argon for 30 min. N-{9-[(2R,4S,5S)-5-[(benzoylsulfanyl)methyl]-4-hydroxyoxolan-2-yl]-9H-purin-6-yl}benzamide (2.0 g, 4.21 mmol) was dissolved in the argon purged solution of THF-MeOH (6:4, 75 mL) under argon and cooled to 0 to −5° C. To this solution was added the NaOH solution (0.5 M, 25.25 mL, 12.62 mmol) and the reaction mixture was stirred at 0 to −5° C. for 30 min. Citric acid was added (3.18 g, 15.14 mmol) at 0° C. A saturated NaHCO₃ solution (70 mL) was added to the reaction mixture and extracted with ethyl acetate (75 mL×3). The combined organic layers were washed with brine, dried over Na₂SO₄, filtered and evaporated under reduced pressure to get N-{9-[(2R,4S,5S)-4-hydroxy-5-(sulfanylmethyl)oxolan-2-yl]-9H-purin-6-yl}benzamide (1.9 g, crude) as colorless viscous oil. (MS: (ESI): m/z=372.1 [M+H]⁺).

To a solution of N-{9-[(2R,4S,5S)-4-hydroxy-5-(sulfanylmethyl)oxolan-2-yl]-9H-purin-6-yl}benzamide (1.9 g, crude, azeotropically distilled with pyridine) in dry pyridine (10 mL, argon purged) was added DMTrCl (1.56 g, 4.60 mmol) at 25° C. and the reaction mixture was stirred at 25° C. for 4 h. Volatiles were removed under reduced pressure to get crude compound. The resulting crude was purified by combiflash (0.5% MeOH in CH₂Cl₂ containing 0.5% triethylamine) to get N-{9-[(2R,4S,5S)-5-({[bis(4-methoxyphenyl)(phenyl)methyl]sulfanyl}methyl)-4-hydroxyoxolan-2-yl]-9H-purin-6-yl}benzamide (1.2 g, 42% after two steps) as off white solid. (MS: (ESI): m/z=674.3 [M+H]⁺).

A solution of 5-ethylmercapto-1H-tetrazole (1.93 g, 14.84 mmol, 0.25 M solution in 60 mL dry acetonitrile) was added to a stirred solution of N-{9-[(2R,4S,5S)-5-({[bis(4-methoxyphenyl)(phenyl)methyl]sulfanyl}methyl)-4-hydroxyoxolan-2-yl]-9H-purin-6-yl}benzamide (5 g, 7.42 mmol) in dry CH₂Cl₂ (120 mL) under argon at 25° C. To the resulting reaction mixture was added 2-cyanoethyl tetra isopropylphosphorodiamidite (4.71 mL, 14.84 mmol) and stirring continued at 25° C. for 4 h. Then the reaction mixture was diluted with CH₂Cl₂ (250 mL) and poured onto a sat. NaHCO₃ solution (250 mL), the organic layer was separated off and the aqueous layer was extracted with CH₂Cl₂ (250 mL×2). The combined organic layers were dried over Na₂SO₄ and concentrated under reduced pressure to get crude compound which was purified by amine silica gel (80% CH₂Cl₂ in hexane) to get impure compound (3.1 g) which was again purified as above to get the desired N-{9-[(2R,4S,5S)-5-({[bis(4-methoxyphenyl)(phenyl)methyl]sulfanyl}methyl)-4-({[bis(propan-2-yl)amino](2-cyanoethoxy)phosphanyl}oxy)oxolan-2-yl]-9H-purin-6-yl}benzamide (5′-Mercapto-DNA adenosine phosphoramidite, 2.32 g, 36%) as white solid. (MS: (ESI): m/z=874.8 [M+H]⁺).

### Example 3

### Incorporation of 3′-S DNA Monomers in Oligonucleotide Synthesis

Oligonucleotides were synthesized using a MerMade 12 automated DNA synthesizer by Bioautomation. Syntheses were conducted on a 1 μmol scale using a controlled pore glass support (500Å) bearing a universal linker.

In standard cycle procedures for the coupling of DNA and LNA phosphoramidites DMT deprotection was performed with 3% (w/v) trichloroacetic acid in CH₂Cl₂ in three applications of 200 μL for 30 sec. The respective phosphoramidites were coupled three times with 100 μL of 0.1M solutions in acetonitrile (or acetonitrile/CH₂Cl₂ 1:1 for the LNA-^{Me}C building block) and 110 μL of a 0.1M solution of 5-(3,5-bis(trifluoromethylphenyl))-1H-tetrazole in acetonitrile as an activator and a coupling time of 180 sec. For thiooxidation a 0.1M solution of 3-amino-1,2,4-dithiazole-5-thione in acetonitrile/pyridine 1:1 was used (3×190 μL, 55 sec). Capping was performed using THF/lutidine/Ac₂O 8:1:1 (CapA, 75 μmol) and THF/N-methylimidazole 8:2 (CapB, 75 μmol) for 55 sec.

Synthesis cycles for the incorporation of 2′,3′-dideoxy-3′-mercapto phosphoramidites included DMT deprotection using 3% (w/v) of trichloroacetic acid in CH₂Cl₂ in three applications of 200 μL for 30 sec. Phosphoramidite coupling was performed ten times with 40 μL of 0.1m solutions in acetonitrile and 44 μL of a 0.1m solution of 5-(3,5-bis(trifluoromethylphenyl))-1H-tetrazole in acetonitrile with a coupling time of 900 sec. Oxidation was performed immediately after coupling by applying six times 200 μL of a 2 mm solution of iodine in THF/H₂O/pyridine 77:2:21 for 50 sec. Capping was performed using THF/lutidine/Ac₂O 8:1:1 (CapA, 75 μmol) and THF/N-methylimidazole 8:2 (CapB, 75 μmol) for 55 sec.

Removal of the nucleobase protecting groups and cleavage from the solid support was achieved under standard conditions using 32% aqueous ammonia at 55° C. for a minimum of 8h. Crude DMT-on oligonucleotides were purified either using a solid phase extraction cartridge or by RP-HPLC purification using a C18 column followed by DMT removal with 80% aqueous acetic acid and ethanol precipitation.

### Example 4

### Incorporation of 5′-S-DNA Monomers in Oligonucleotide Synthesis

Oligonucleotides were synthesized using a MerMade 12 automated DNA synthesizer by Bioautomation. Syntheses were conducted on a 1 μmol scale using a controlled pore glass support (500Å) bearing a universal linker.

In standard cycle procedures for the coupling of DNA and LNA phosphoramidites DMT deprotection was performed with 3% (w/v) trichloroacetic acid in CH₂Cl₂ in three applications of 200 μL for 30 sec. The respective phosphoramidites were coupled three times with 100 μL of 0.1m solutions in acetonitrile (or acetonitrile/CH₂Cl₂ 1:1 for the LNA-^{Me}C building block) and 110 μL of a 0.1M solution of 5-(3,5-bis(trifluoromethylphenyl))-1H-tetrazole in acetonitrile as an activator and a coupling time of 180 sec. For thiooxidation a 0.1M solution of 3-amino-1,2,4-dithiazole-5-thione in acetonitrile/pyridine 1:1 was used (3×190 μL, 55 sec). Capping was performed using THF/lutidine/Ac₂O 8:1:1 (CapA, 75 μmol) and THF/N-methylimidazole 8:2 (CapB, 75 μmol) for 55 sec.

Synthesis cycles for the incorporation of 2′,5′-dideoxy-5′-mercapto phosphoramidites included coupling of the phosphoramidite building blocks using 100 μL of 0.1m solutions in acetonitrile and 110 μL of a 0.1m solution of 5-(3,5-bis(trifluoromethylphenyl))-1H-tetrazole in acetonitrile with a coupling time of 180 sec. Triple couplings were performed. Depending on the desired sequence the synthesis column was subjected either to thiooxidation using a 0.1m solution of 3-amino-1,2,4-dithiazole-5-thione in acetonitrile/pyridine 1:1 (3×190 μL, 55 sec) or oxidation using a 2 mM solution of iodine in THF/H₂O/pyridine 77:2:21 (6×200 μL, 55 sec). Capping was performed using THF/lutidine/Ac₂O 8:1:1 (CapA, 75 μmol) and THF/N-methylimidazole 8:2 (CapB, 75 μmol) for 55 sec. DMT deprotection and liberation of the thiol was conducted with 3% (w/v) trichloroacetic acid in CH₂Cl₂ in 15 applications of 200 μL for 30 sec. After coupling of the subsequent phosphoramidite building block according to the conditions given above, oxidation was performed using a 2 mm solution of iodine in THF/H₂O/pyridine 77:2:21 (6×200 μL, 55 sec).

DMT deprotection and liberation of the thiol was also advantageously conducted with 3-6 applications of 200 μL for 45 sec of 1-10% (v/v) trifluoroacetic acid or 5-10% (w/v) trichloroacetic acid, in the presence of 5-30% (v/v) triethylsilane in CH₂Cl₂ and/or 2-10% of p-methoxy thiophenol in CH₂Cl₂.

Removal of the nucleobase protecting groups and cleavage from the solid support was achieved under standard conditions using 32% aqueous ammonia at 55° C. for a minimum of 8 h. Crude DMT-on oligonucleotides were purified either using a solid phase extraction cartridge or by RP-HPLC purification using a C18 column followed by DMT removal with 80% aqueous acetic acid and ethanol precipitation.

### Example 5

### Incorporation of 5′-S-LNA Monomers in Oligonucleotide Synthesis

Oligonucleotides were synthesized using a MerMade 12 automated DNA synthesizer by Bioautomation. Syntheses were conducted on a 1 μmol scale using a controlled pore glass support (500 Å) bearing a universal linker.

In standard cycle procedures for the coupling of DNA and LNA phosphoramidites DMT deprotection was performed with 3% (w/v) trichloroacetic acid in CH₂Cl₂ in three applications of 200 μL for 30 sec. The respective phosphoramidites were coupled three times with 100 μL of 0.1 m solutions in acetonitrile (or acetonitrile/CH₂Cl2 1:1 for the LNA-^{Me}C building block) and 110 μL of a 0.1m solution of 5(3,5-bis(trifluoromethylphenyl))-1H-tetrazole in acetonitrile as an activator and a coupling time of 180 sec. For thiooxidation a 0.1M solution of 3-amino-1,2,4-dithiazole-5-thione in acetonitrile/pyridine 1:1 was used (3×190 μL, 55 sec). Capping was performed using THF/lutidine/Ac₂O 8:1:1 (CapA, 75 μmol) and THF/N-methylimidazole 8:2 (CapB, 75 μmol) for 55 sec.

Synthesis cycles for the incorporation of 2′,5′-dideoxy-5′-mercapto LNA-phosphoramidites included coupling of the phosphoramidite building blocks using 100 μL of 0.1M solutions in acetonitrile and 110 μL of a 0.1M solution of 5-(3,5-bis(trifluoromethylphenyl))-1H-tetrazole in acetonitrile with a coupling time of 600 sec. Triple couplings were performed. Depending on the desired sequence the synthesis column was subjected either to thiooxidation using a 0.1M solution of 3-amino-1,2,4-dithiazole-5-thione in acetonitrile/pyridine 1:1 (3×190 μL, 55 sec) or oxidation using a 2 mM solution of iodine in THF/H₂O/pyridine 77:2:21 (6×200 μL, 55 sec). Capping was performed using THF/lutidine/Ac₂O 8:1:1 (CapA, 75 μmol) and THF/N-methylimidazole 8:2 (CapB, 75 μmol) for 55 sec. DMT deprotection and liberation of the thiol was conducted with 3% (w/v) trichloroacetic acid in CH₂Cl₂ in 15 applications of 200 μL for 30 sec. After coupling of the subsequent phosphoramidite building block according to the conditions given above, oxidation was performed using a 2 mm solution of iodine in THF/H₂O/pyridine 77:2:21 (6×200 μL, 55 sec).

DMT deprotection and liberation of the thiol was also advantageously conducted with 3-6 applications of 200 μL for 45 sec of 1-10% (v/v) trifluoroacetic acid or 5-10% (w/v) trichloroacetic acid, in the presence of 5-30% (v/v) triethylsilane in CH₂Cl₂ and/or 2-10% of p-methoxy thiophenol in CH₂Cl₂.

Removal of the nucleobase protecting groups and cleavage from the solid support was achieved under standard conditions using 32% aqueous ammonia at 55° C. for a minimum of 8 h. Crude DMT-on oligonucleotides were purified either using a solid phase extraction cartridge or by RP-HPLC purification using a C18 column followed by DMT removal with 80% aqueous acetic acid and ethanol precipitation.

### Example 6

### 3′-S Oligonucleotides

According to the General Procedure outlined above the following molecules were prepared:

| Compound | | Calculated | Found |
|---|---|---|---|
| ID No. | Sequence | mass | mass |
| #1 | GAGttacttgccaA^{m}CT | 5275.4903 | 5275.4745 |
| #2 | GAGttacttgccaA^{m}CT | 5275.4903 | 5275.4760 |
| #3 | GAGttacttgccaA^{m}CT | 5275.4903 | 5275.4826 |
| #4 | GAGttacttgccaA^{m}CT | 5275.4903 | 5275.4858 |
| #5 | GAGttacttgccaA^{m}CT | 5275.4903 | 5275.4706 |
| #6 | GAGttacttgccaA^{m}CT | 5275.4903 | 5275.4716 |
| #7 | GAGttacttgccaA^{m}CT | 5275.4903 | 5275.4743 |
| #8 | GAGttacttgccaA^{m}CT | 5275.4903 | 5275.4892 |
| #9 | GAGttacttgccaA^{m}CT | 5275.4903 | 5275.4947 |
| #10 | GAGttacttgccaA^{m}CT | 5275.4903 | 5275.4845 |
| #11 | GAGttacttgccaA^{m}CT | 5275.4903 | 5275.5082 |
| #12 | GAGttacttgccaA^{m}CT | 5275.4903 | 5275.4948 |
| #13 | G^{m}CattggtatT^{m}CA | 4322.4292 | 4322.4244 |
| #14 | G^{m}CattggtatT^{m}CA | 4322.4292 | 4322.4262 |
| #15 | G^{m}CattggtatT^{m}CA | 4322.4292 | 4322.2726 |
| #16 | G^{m}CattggtatT^{m}CA | 4322.4292 | 4322.2687 |
| #17 | G^{m}CattggtatT^{m}CA | 4322.4292 | 4322.4256 |
| #18 | G^{m}CattggtatT^{m}CA | 4322.4292 | 4322.4278 |
| #19 | G^{m}CattggtatT^{m}CA | 4322.4292 | 4322.4266 |
| #20 | G^{m}CattggtatT^{m}CA | 4322.4292 | 4322.4263 |
| #21 | G^{m}CattggtatT^{m}CA | 4322.4292 | 4322.4515 |
| #22 | A^{m}CTtatggtta^{m}CG | 4322.4292 | 4322.4214 |
| #23 | A^{m}CTtatggtta^{m}CG | 4322.4292 | 4322.4224 |
| #24 | A^{m}CTtatggtta^{m}CG | 4322.4292 | 4322.4220 |
| #25 | A^{m}CTtatggtta^{m}CG | 4322.4292 | 4322.4560 |
| #26 | A^{m}CTtatggtta^{m}CG | 4322.4292 | 4322.4490 |
| #27 | A^{m}CTtatggtta^{m}CG | 4322.4292 | 4322.4208 |
| #28 | A^{m}CTtatggtta^{m}CG | 4322.4292 | 4322.4210 |
| #29 | A^{m}CTtatggtta^{m}CG | 4322.4292 | 4322.4224 |
| #30 | ^{m}CA^{m}Cattccttgct^{m}CTG | 5230.4927 | 5230.4872 |
| #31 | ^{m}CA^{m}Cattccttgct^{m}CTG | 5230.4927 | 5230.4877 |
| #32 | ^{m}CA^{m}Cattccttgct^{m}CTG | 5230.4927 | 5230.4872 |
| #33 | ^{m}CA^{m}Cattccttgct^{m}CTG | 5230.4927 | 5230.4903 |
| #34 | ^{m}CA^{m}Cattccttgct^{m}CTG | 5230.4927 | 5230.4862 |
| #35 | ^{m}CA^{m}Cattccttgct^{m}CTG | 5230.4927 | 5230.4898 |
| #36 | ^{m}CA^{m}Cattccttgct^{m}CTG | 5230.4927 | 5230.4872 |
| #37 | ^{m}CA^{m}Cattccttgct^{m}CTG | 5230.4927 | 5230.4829 |
| #38 | ^{m}CA^{m}Cattccttgct^{m}CTG | 5230.4927 | 5230.4904 |
| #39 | ^{m}CA^{m}Cattccttgct^{m}CTG | 5230.4927 | 5230.4879 |

a, g, c, t represent 2′,3′-Dideoxy-3′-mercapto modifications
A, G, ^{m}C, T represent LNA nucleotides
a, g, c, t represent DNA nucleotides
all oligonucleotides were prepared as full phosphorothioates (with a sulfur atom either at a terminal or in the 3′-position)

### Example 7

### 5′-S Oligonucleotides

According to the General Procedure outlined above the following molecules were prepared:

| Compound | | Calculated | Found |
|---|---|---|---|
| ID No. | Sequence | mass | mass |
| #40 | GAGttacttgccaA^{m}CT | 5275.4903 | 5275.4880 |
| #41 | GAGttacttgccaA^{m}CT | 5275.4903 | 5275.4849 |
| #42 | GAGttacttgccaA^{m}CT | 5275.4903 | 5275.5014 |
| #43 | GAGttacttgccaA^{m}CT | 5275.4903 | 5275.4904 |
| #44 | G^{m}CattggtatT^{m}CA | 4322.4292 | 4322.4227 |
| #45 | G^{m}CattggtatT^{m}CA | 4322.4292 | 4322.4236 |
| #46 | G^{m}CattggtatT^{m}CA | 4322.4292 | 4322.4238 |
| #47 | G^{m}CattggtatT^{m}CA | 4322.4292 | 4322.4228 |
| #48 | G^{m}CattggtatT^{m}CA | 4322.4292 | 4322.4235 |
| #49 | G^{m}CattggtatT^{m}CA | 4322.4292 | 4322.4225 |
| #50 | G^{m}CattggtatT^{m}CA | 4322.4292 | 4322.4226 |
| #51 | G^{m}CattggtatT^{m}CA | 4322.4292 | 4322.4220 |
| #52 | G^{m}CattggtatT^{m}CA | 4322.4292 | 4322.4256 |

a, g, c, t represent 2′,5′-Dideoxy-5′-mercapto modifications
A, G, ^{m}C, T represent LNA nucleotides
a, g, c, t represent DNA nucleotides
all oligonucleotides were prepared as full phosphorothioates (with a sulfur atom either at a terminal or in the 5′-position)

1. A process for the manufacture of an oligonucleotide comprising at least one non-chiral phosphorothioate internucleoside linkage of formula (I) 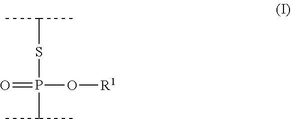
said process comprising the step of reacting an oligonucleotide comprising an internucleoside linkage of formula (II) 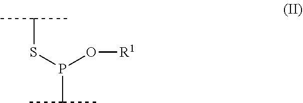
in the presence of iodine, wherein the concentration of iodine is between about 0.001 M and about 0.01 M; and
wherein R¹ is a phosphate protecting group.

2. A process according to claim 1, wherein the sulfur atom of the at least one non-chiral phosphorothioate internucleotide linkage of formula (I) or (II) is linked to the 3′ carbon atom or 5′ carbon atom of an adjacent nucleoside of the oligonucleotide.

3. A process according to claim 1, wherein the oligonucleotide comprising at least one non-chiral phosphorothioate internucleoside linkage of formula (I) comprises a fragment of formula (III) 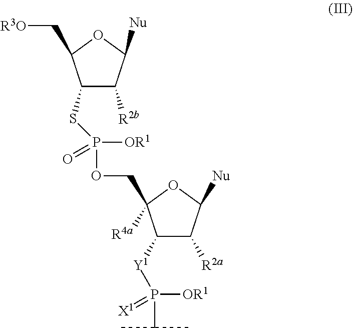
wherein
X¹ is oxygen or sulfur;
Y¹ is oxygen or sulfur;
provided that X¹ and Y¹ are not both sulfur at the same time;
each R¹ is independently is a phosphate protecting group;
R^{2a} is hydrogen, hydroxyl, fluoro, alkyl, alkoxy, alkoxyalkoxy, —NH₂, alkylamino, dialkylamino, alkylcarbonylamino, azido, —SH, —CN, —CF₃, —OCF₃, alkyl sulfanylalkoxy, aminooxyalkoxy, alkylaminooxyalkoxy, dialkylaminooxyalkoxy, aminocarbonylalkoxy, alkylaminocarbonylalkoxy or dialkylaminocarbonylalkoxy;
R^{4a} is hydrogen or hydroxyalkyl;
or R^{2a} and R^{4a} together form —CH₂O—, —CH₂NH—, —CH₂S—, —CH₂N(OR^{p})—, —CHCH₃O—, —C(CH₃)₂O—, —CH₂C(═CH₂)—, —CHCH₃C(═CH₂)—, —CHCH₃S—, —CH₂NR^{p}—, CH₂CH₂O—, —CH₂CH₂CH₂O—, —CH₂OCH₂—, —CH(CH₂OCH₃)O—, CH(CH₂CH₃)O— or —CH₂OCH₂O—;
provided that when Y¹ is sulfur, then R^{4a} is hydrogen;
R^{2b} is hydrogen, hydroxyl, fluoro, alkyl, alkoxy, alkoxyalkoxy, —NH₂, alkylamino, dialkylamino, alkylcarbonylamino, azido, —SH, —CN, —CF₃, —OCF₃, alkyl sulfanylalkoxy, aminooxyalkoxy, alkylaminooxyalkoxy, dialkylaminooxyalkoxy, aminocarbonylalkoxy, alkylaminocarbonylalkoxy or dialkylaminocarbonylalkoxy;
R³ is a hydroxyl protecting group;
each R^{p} is independently alkyl; and
each Nu is independently a nucleobase.

4. A process according to claim 1, wherein the oligonucleotide comprising an internucleoside linkage of formula (II) comprises a fragment of formula (IV) 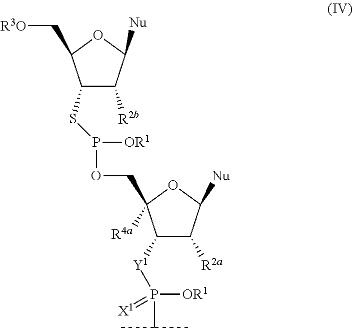
wherein
X¹ is oxygen or sulfur;
Y¹ is oxygen or sulfur;
provided that X¹ and Y¹ are not both sulfur at the same time;
each R¹ is independently is a phosphate protecting group;
R^{2a} is hdydrogen, hydroxyl, fluoro, alkyl, alkoxy, alkoxyalkoxy, alkylamino, dialkylamino, alkylcarbonylamino, azido, —SH, —CN, —OCF, alkyl sulfanylalkoxy, aminooxyalkoxy, alkylaminooxyalkoxy, dialkylaminooxyalkoxy, aminocarbonylalkoxy, alkylaminocarbonylalkoxy or dialkylaminocarbonylalkoxy;
R^{4a} is hydrogen or hydroxyalkyl;
or R^{2a} and R^{4a} together form —CH₂O—, —CH₂NH—, —CH₂S—, —CH₂N(OR^{p})—, —CHCH₃O—, —C(CH₃)₂O—, —CH₂C(═CH)—, —CHCH₃C(═CH)—, —CHCH₃S—, —CH₂NR^{p}—, CH₂CH₂O—, —CH₂CH₂CH₂O—, —CH₂OCH₂—, —CH(CH₂OCH)O—, CH(CH₂CH)O— or —CH₂OCH₂O—;
provided that when Y¹ is sulfur, then R^{4a} is hydrogen;
R^{2b} is hydrogen, hydroxyl, fluoro, alkyl, alkoxy, alkoxyalkoxy, alkylamino, dialkylamino, alkylcarbonylamino, azido, —SH, —CN, —OCF, alkyl sulfanylalkoxy, aminooxyalkoxy, alkylaminooxyalkoxy, dialkylaminooxyalkoxy, aminocarbonylalkoxy, alkylaminocarbonylalkoxy or dialkylaminocarbonylalkoxy;
R³ is a hydroxyl protecting group;
each R^{p} is independently alkyl; and
each Nu is independently a nucleobase.

5. A process according claim 3, wherein the oligonucleotide comprising at least one non-chiral phosphorothioate internucleoside linkage of formula (I) is reacted in the presence of acid to arrive at an oligonucleotide comprising a fragment of formula (V) 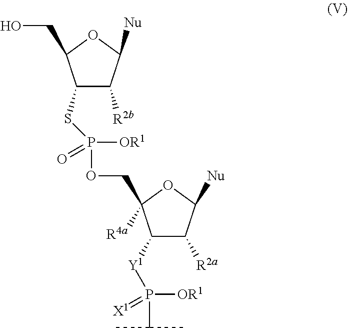
wherein
X¹ is oxygen or sulfur;
Y¹ is oxygen or sulfur;
provided that X¹ and Y¹ are not both sulfur at the same time;
each R¹ is independently is a phosphate protecting group;
R^{2a} is hydrogen, hydroxyl, fluoro, alkyl, alkoxy, alkoxyalkoxy, —NH₂, alkylamino, dialkylamino, alkylcarbonylamino, azido, —SH, —CN, —CF₃, —OCF₃, alkyl sulfanylalkoxy, aminooxyalkoxy, alkylaminooxyalkoxy, dialkylaminooxyalkoxy, aminocarbonylalkoxy, alkylaminocarbonylalkoxy or dialkylaminocarbonylalkoxy;
R^{4a} is hydrogen or hydroxyalkyl;
or R^{2a} and R^{4a} together form —CH₂O—, —CH₂NH—, —CH₂S—, —CH₂N(OR^{p})—, —CHCH₃O—, —C(CH₃)₂O—, —CH₂C(═CH₂)—, —CHCH₃C(═CH₂)—, —CHCH₃S—, —CH₂NR^{p}—, CH₂CH₂O—, —CH₂CH₂CH₂O—, —CH₂OCH₂—, —CH(CH₂OCH₃)O—, CH(CH₂CH₃)O— or —CH₂OCH₂O—;
provided that when Y¹ is sulfur, then R^{4a} is hydrogen;
R^{2b} is hydrogen, hydroxyl, fluoro, alkyl, alkoxy, alkoxyalkoxy, alkylamino, dialkylamino, alkylcarbonylamino, azido, —SH, —CN, —CF₃, —OCF₃, alkyl sulfanylalkoxy, aminooxyalkoxy, alkylaminooxyalkoxy, dialkylaminooxyalkoxy, aminocarbonylalkoxy, alkylaminocarbonylalkoxy or dialkylaminocarbonylalkoxy;
R³ is a hydroxyl protecting group;
each R^{p} is independently alkyl; and
each Nu is independently a nucleobase.

6. A process according to claim 5, wherein the oligonucleotide comprising a fragment of formula (V) is reacted in the presence of a compound of formula (VI) 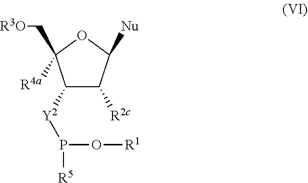
to arrive at an oligonucleotide comprising at least one non-chiral phosphorothioate internucleoside linkage of formula (I) comprising a fragment of formula (VII) 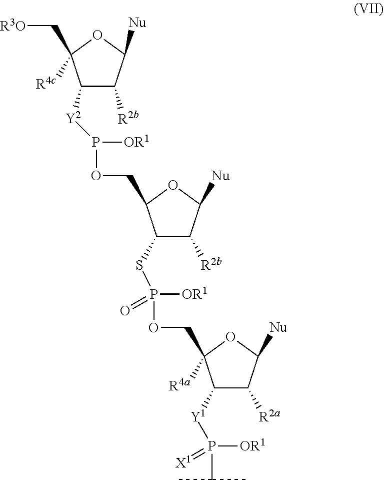
wherein
Y² is oxygen or sulfur;
R^{2c} is hydrogen, hydroxyl, fluoro, alkyl, alkoxy, alkoxyalkoxy, —NH₂, alkylamino, dialkylamino, alkylcarbonylamino, azido, —SH, —CN, —CF₃, —OCF₃, alkyl sulfanylalkoxy, aminooxyalkoxy, alkylaminooxyalkoxy, dialkylaminooxyalkoxy, aminocarbonylalkoxy, alkylaminocarbonylalkoxy or dialkylaminocarbonylalkoxy;
R^{4c} is hydrogen or hydroxyalkyl;
or R^{2c} and R^{4c} together form —CH₂O—, —CH₂NH—, —CH₂S—, —CH₂N(OR^{p})—, —CHCH₃O—, —C(CH₃)₂O—, —CH₂C(═CH₂)—, —CHCH₃C(═CH₂)—, —CHCH₃S—, —CH₂NR^{p}—, CH₂CH₂O—, —CH₂CH₂CH₂O—, —CH₂OCH₂—, —CH(CH₂OCH₃)O—, CH(CH₂CH₃)O— or —CH₂OCH₂O—;
provided that when Y² is sulfur, then R⁴⁰ is hydrogen;
R⁵ is dialkylamino;
each R^{p} is independently alkyl;
X¹ is oxygen or sulfur;
Y¹ is oxygen or sulfur;
provided that X¹ and Y¹ are not both sulfur at the same time;
each R¹ is independently is a phosphate protecting group;
R^{2a} is hydrogen, hydroxyl, fluoro, alkyl, alkoxy, alkoxyalkoxy, alkylamino, dialkylamino, alkylcarbonylamino, azido, —SH, —CN, —OCF, alkyl sulfanylalkoxy, aminooxyalkoxy, alkylaminooxyalkoxy, dialkylaminooxyalkoxy, aminocarbonylalkoxy, alkylaminocarbonylalkoxy or dialkylaminocarbonylalkoxy;
R^{4a} is hydrogen or hydroxyalkyl;
or R^{2a} and R^{4a} together form —CH₂O—, —CH₂NH—, —CH₂S—, —CH₂N(OR^{p})—, —CHCH₃O—, —C(CH₃)₂O—, —CH₂C(═CH)—, —CHCH₃C(═CH)—, —CHCH₃S—, —CH₂NR^{p}—, CH₂CH₂O—, —CH₂CH₂CH₂O—, —CH₂OCH₂—, —CH(CH₂OCH)O—, CH(CH₂CH)O— or —CH₂OCH₂O—;
provided that when Y¹ is sulfur, then R^{4a} is hydrogen;
R^{2b} is hydrogen, hydroxyl, fluoro, alkyl, alkoxy, alkoxyalkoxy, alkylamino, dialkylamino, alkylcarbonylamino, azido, —SH, —CN, —OCF, alkyl sulfanylalkoxy, aminooxyalkoxy, alkylaminooxyalkoxy, dialkylaminooxyalkoxy, aminocarbonylalkoxy, alkylaminocarbonylalkoxy or dialkylaminocarbonylalkoxy;
R³ is a hydroxyl protecting group;
each R^{p} is independently alkyl; and
each Nu is independently a nucleobase

7. A process according to claim 6, wherein the oligonucleotide comprising a fragment of formula (VII) is reacted in the presence of a thiooxidation agent or iodine, wherein the concentration of iodine is between about 0.001 M and about 0.01 M, to arrive at an oligonucleotide comprising at least one non-chiral phosphorothioate internucleoside linkage of formula (I) comprising a fragment of formula (VIII) 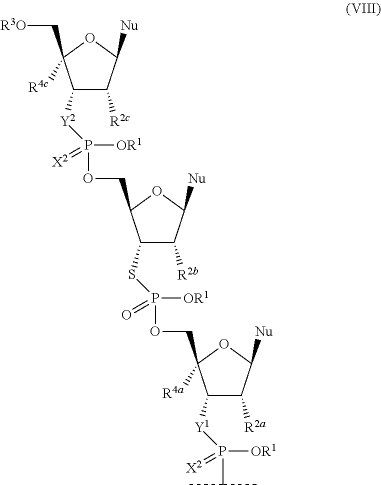
wherein
X² is oxygen or sulfur;
Y² is oxygen or sulfur;
provided that X² and Y² are not both sulfur at the same time;
X¹ is oxygen or sulfur;
Y¹ is oxygen or sulfur;
provided that X¹ and Y¹ are not both sulfur at the same time;
each R¹ is independently is a phosphate protecting group;
R^{2a} is hydrogen, hydroxyl, fluoro, alkyl, alkoxy, alkoxyalkoxy, alkylamino, dialkylamino, alkylcarbonylamino, azido, —SH, —CN, —CF₃, —OCF₃, alkyl sulfanylalkoxy, aminooxyalkoxy, alkylaminooxyalkoxy, dialkylaminooxyalkoxy, aminocarbonylalkoxy, alkylaminocarbonylalkoxy or dialkylaminocarbonylalkoxy;
R^{4a} is hydrogen or hydroxyalkyl;
or R^{2a} and R^{4a} together form —CH₂O—, —CH₂NH—, —CH₂S—, —CH₂N(OR^{p})—, —CHCH₃O—, —C(CH₃)₂O—, —CH₂C(═CH)—, —CHCH₃C(═CH)—, —CHCH₃S—, —CH₂NR^{p}—, —CH₂CH₂O—, —CH₂CH₂CH₂O—, —CH₂OCH₂—, —CH(CH₂OCH)O—, —CH(CH₂CH)O— or —CH₂OCH₂O—;
provided that when Y¹ is sulfur, then R^{4a} is hydrogen;
R^{2b} is hydrogen, hydroxyl, fluoro, alkyl, alkoxy, alkoxyalkoxy, alkylamino, dialkylamino, alkylcarbonylamino, azido, —SH, —CN, —OCF, alkyl sulfanylalkoxy, aminooxyalkoxy, alkylaminooxyalkoxy, dialkylaminooxyalkoxy, aminocarbonylalkoxy, alkylaminocarbonylalkoxy or dialkylaminocarbonylalkoxy;
R³ is a hydroxyl protecting group;
each R^{p} is independently alkyl; and
each Nu is independently a nucleobase
R^{2c} is hydrogen, hydroxyl, fluoro, alkyl, alkoxy, alkoxyalkoxy, alkylamino, dialkylamino, alkylcarbonylamino, azido, —SH, —CN, —CF₃, —OCF₃, alkyl sulfanylalkoxy, aminooxyalkoxy, alkylaminooxyalkoxy, dialkylaminooxyalkoxy, aminocarbonylalkoxy, alkylaminocarbonylalkoxy or dialkylaminocarbonylalkoxy, and
R^{4c} is hydrogen or hydroxyalkyl
and R^{2c} and R^{4c} are as defined in claim 6.

8. A process according to claim 7, wherein the oligonucleotide comprising a fragment of formula (VII) is reacted in the presence of a thiooxidation agent when Y² is oxygen.

9. A process according to claim 7, wherein the oligonucleotide comprising a fragment of formula (VII) is reacted in the presence of iodine when Y² is sulfur.

10. A process according to claim 1, wherein the oligonucleotide comprising at least one non-chiral phosphorothioate internucleoside linkage of formula (I) comprises a fragment of formula (IX) 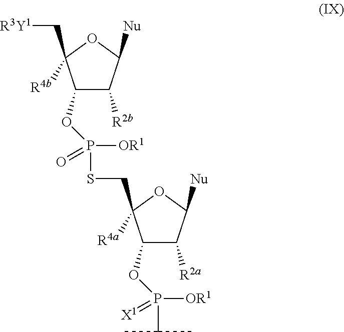
wherein
X¹ is oxygen or sulfur;
Y¹ is oxygen or sulfur;
each R¹ is independently is a phosphate protecting group;
R^{2a} is hydrogen, hydroxyl, fluoro, alkyl, alkoxy, alkoxyalkoxy, —NH₂, alkylamino, dialkylamino, alkylcarbonylamino, azido, —SH, —CN, —CF₃, —OCF₃, alkyl sulfanylalkoxy, aminooxyalkoxy, alkylaminooxyalkoxy, dialkylaminooxyalkoxy, aminocarbonylalkoxy, alkylaminocarbonylalkoxy or dialkylaminocarbonylalkoxy;
R^{4a} is hydrogen or hydroxyalkyl;
or R^{2a} and R^{4a} together form —CH₂O—, —CH₂NH—, —CH₂S—, —CH₂N(OR^{p})—, —CHCH₃O—, —C(CH₃)₂O—, —CH₂C(═CH₂)—, —CHCH₃C(═CH₂)—, —CHCH₃S—, —CH₂NR^{p}—, —CH₂CH₂O—, —CH₂CH₂CH₂O—, —CH₂OCH₂—, —CH(CH₂OCH₃)O—, —CH(CH₂CH₃)O— or —CH₂OCH₂O—;
R^{2b} is hydrogen, hydroxyl, fluoro, alkyl, alkoxy, alkoxyalkoxy, —NH₂, alkylamino, dialkylamino, alkylcarbonylamino, azido, —SH, —CN, —CF₃, —OCF₃, alkyl sulfanylalkoxy, aminooxyalkoxy, alkylaminooxyalkoxy, dialkylaminooxyalkoxy, aminocarbonylalkoxy, alkylaminocarbonylalkoxy or dialkylaminocarbonylalkoxy;
R^{4b} is hydrogen or hydroxyalkyl;
or R^{2b} and R^{4b} together form —CH₂O—, —CH₂NH—, —CH₂S—, —CH₂N(OR^{p})—, —CHCH₃O—, —C(CH₃)₂O—, —CH₂C(═CH₂)—, —CHCH₃C(═CH₂)—, —CHCH₃S—, —CH₂NR^{p}—, —CH₂CH₂O—, —CH₂CH₂CH₂O—, —CH₂OCH₂—, —CH(CH₂OCH₃)O—, —CH(CH₂CH₃)O— or —CH₂OCH₂O—;
R³ is a hydroxyl protecting group or a thiohydroxyl protecting group;
each R^{p} is independently alkyl; and
each Nu is independently a nucleobase.

11. A process according to claim 1, wherein the oligonucleotide comprising an internucleoside linkage of formula (II) comprises a fragment of formula (X) 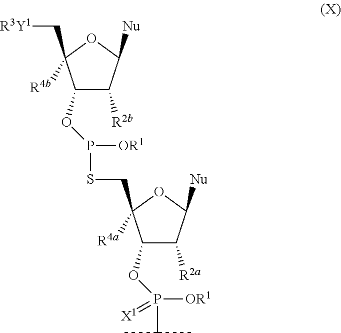
wherein
X¹ is oxygen or sulfur;
Y¹ is oxygen or sulfur;
each R¹ is independently is a phosphate protecting group;
R^{2a} is hydrogen, hydroxyl, fluoro, alkyl, alkoxy, alkoxyalkoxy, —NH₂, alkylamino, dialkylamino, alkylcarbonylamino, azido, —SH, —CN, —CF₃, —OCF₃, alkyl sulfanylalkoxy, aminooxyalkoxy, alkylaminooxyalkoxy, dialkylaminooxyalkoxy, aminocarbonylalkoxy, alkylaminocarbonylalkoxy or dialkylaminocarbonylalkoxy;
R^{4a} is hydrogen or hydroxyalkyl;
or R^{2a} and R^{4a} together form —CH₂O—, —CH₂NH—, —CH₂S—, —CH₂N(OR')—, —CHCH₃O—, —C(CH₃)₂O—, —CH₂C(═CH₂)—, —CHCH₃C(═CH₂)—, —CHCH₃S—, —CH₂NR^{p}—, —CH₂CH₂O—, —CH₂CH₂CH₂O—, —CH₂OCH₂—, —CH(CH₂OCH)O—, —CH(CH₂CH)O— or —CH₂OCH₂O—;
R^{2b} is hydrogen, hydroxyl, fluoro, alkyl, alkoxy, alkoxyalkoxy, alkylamino, dialkylamino, alkylcarbonylamino, azido, —SH, —CN₃, CF₃, —OCF₃, alkyl sulfanylalkoxy, aminooxyalkoxy, alkylaminooxyalkoxy, dialkylaminooxyalkoxy, aminocarbonylalkoxy, alkylaminocarbonylalkoxy or dialkylaminocarbonylalkoxy;
R^{4b} is hydrogen or hydroxyalkyl;
or R^{2b} and R^{4b} together form —CH₂O—, —CH₂NH—, —CH₂S—, —CH₂N(OR^{p})—, —CHCH₃O—, —C(CH₃)₂O—, —CH₂C(═CH)—, —CHCH₃C(═CH)—, —CHCH₃S—, —CH₂NR^{p}—, —CH₂CH₂O—, —CH₂CH₂CH₂O—, —CH₂OCH₂—, —CH(CH₂OCH)O—, —CH(CH₂CH)O— or —CH₂OCH₂O—;
R³ is a hydroxyl protecting group or a thiohydroxyl protecting group;
each R^{p} is independently alkyl; and
each Nu is independently a nucleobase.

12. A process according to claim 10, wherein the oligonucleotide comprising at least one non-chiral phosphorothioate internucleoside linkage of formula (I) is reacted in the presence of acid to arrive at an oligonucleotide comprising a fragment of formula (XI) 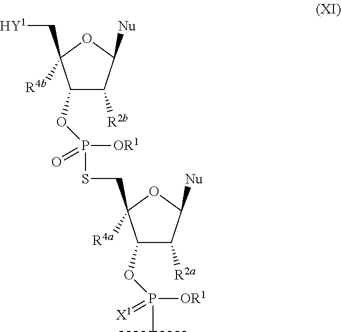
wherein
X¹ is oxygen or sulfur;
Y¹ is oxygen or sulfur;
each R¹ is independently is a phosphate protecting group;
R^{2a} is hydrogen, hydroxyl, fluoro, alkyl, alkoxy, alkoxyalkoxy, —NH₂, alkylamino, dialkylamino, alkylcarbonylamino, azido, —SH, —CN, —CF₃, —OCF₃, alkyl sulfanylalkoxy, aminooxyalkoxy, alkylaminooxyalkoxy, dialkylaminooxyalkoxy, aminocarbonylalkoxy, alkylaminocarbonylalkoxy or dialkylaminocarbonylalkoxy;
R^{4a} is hydrogen or hydroxyalkyl;
or R^{2a} and R^{4a} together form —CH₂O—, —CH₂NH—, —CH₂S—, —CH₂N(OR^{p})—, —CHCH₃O—, —C(CH₃)₂O—, —CH₂C(═CH₂)—, —CHCH₃C(═CH₂)—, —CHCH₃S—, —CH₂NR^{p}—, —CH₂CH₂O—, —CH₂CH₂CH₂O—, —CH₂OCH₂—, —CH(CH₂OCH₃)O—, —CH(CH₂CH₃)O— or —CH₂OCH₂O—;
R^{2b} is hydrogen, hydroxyl, fluoro, alkyl, alkoxy, alkoxyalkoxy, —NH₂, alkylamino, dialkylamino, alkylcarbonylamino, azido, —SH, —CN, —CF₃, —OCF₃, alkyl sulfanylalkoxy, aminooxyalkoxy, alkylaminooxyalkoxy, dialkylaminooxyalkoxy, aminocarbonylalkoxy, alkylaminocarbonylalkoxy or dialkylaminocarbonylalkoxy;
R^{4b} is hydrogen or hydroxyalkyl;
or R^{2b} and R^{4b} together form —CH₂O—, —CH₂NH—, —CH₂S—, —CH₂N(OR^{p})—, —CHCH₃O—, —C(CH₃)₂O—13 , —CH₂C(═CH)—, —CHCH₃C(═CH)—, —CHCH₃S—, —CH₂NR^{p}—, CH₂CH₂O—, —CH₂CH₂CH₂O—, —CH₂OCH₂—, —CH(CH₂OCH)O—, CH(CH₂CH)O— or —CH₂OCH₂O—;
R³ is a hydroxyl protecting group or a thiohydroxyl protecting group;
each R^{p} is independently alkyl; and
each Nu is independently a nucleobase.

13. A process according to claim 12, wherein the oligonucleotide comprising a fragment of formula (XI) is reacted in the presence of a compound of formula (XII) 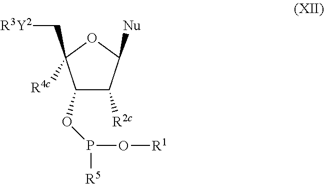
to arrive at an oligonucleotide comprising at least one non-chiral phosphorothioate internucleoside linkage of formula (I) comprising a fragment of formula (XIII) 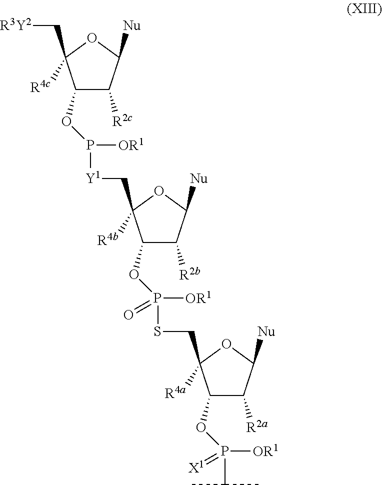
wherein
Y² is oxygen or sulfur;
R^{2c} is hydrogen, hydroxyl, fluoro, alkyl, alkoxy, alkoxyalkoxy, —NH₂, alkylamino, dialkylamino, alkylcarbonylamino, azido, —SH, —CN, —CF₃, —OCF₃, alkyl sulfanylalkoxy, aminooxyalkoxy, alkylaminooxyalkoxy, dialkylaminooxyalkoxy, aminocarbonylalkoxy, alkylaminocarbonylalkoxy or dialkylaminocarbonylalkoxy;
R^{4c} is hydrogen or hydroxyalkyl;
or R^{2c} and R^{4c} together form —CH₂O—, —CH₂NH—, —CH₂S—, —CH₂N(OR^{p})—, —CHCH₃O—, —C(CH₃)₂O—13 , —CH₂C(═CH₂)—, —CHCH₃C(═CH₂)—, —CHCH₃S—, —CH₂NR^{p}—, —CH₂CH₂O—, —CH₂CH₂CH₂O—, —CH₂OCH₂—, —CH(CH₂OCH₃)O—, —CH(CH₂CH₃)O— or —CH₂OCH₂O—;
R³ is a hydroxyl protecting group or a thiohydroxyl protecting group;
R⁵ is dialkylamino;
each R^{p} is independently alkyl; and
X¹ is oxygen or sulfur;
Y¹ is oxygen or sulfur;
each R¹ is independently is a phosphate protecting group;
R^{2a} is hydrogen, hydroxyl, fluoro, alkyl, alkoxy, alkoxyalkoxy, alkylamino, dialkylamino, alkylcarbonylamino, azido, —SH, —CN, —CF₃, —OCF₃, alkyl sulfanylalkoxy, aminooxyalkoxy, alkylaminooxyalkoxy, dialkylaminooxyalkoxy, aminocarbonylalkoxy, alkylaminocarbonylalkoxy or dialkylaminocarbonylalkoxy;
R^{4a} is hydrogen or hydroxyalkyl;
or R^{2a} and R^{4a} together form —CH₂O—, —CH₂NH—, —CH₂S—, —CH₂N(OR^{p})—, —CHCH₃O—, —C(CH₃)₂O—, —CH₂C(═CH)—, —CHCH₃C(═CH)—, —CHCH₃S—, —CH₂NR^{p}—, —CH₂CH₂O—, —CH₂CH₂CH₂O—, —CH₂OCH₂—, —CH(CH₂OCH)O—, —CH(CH₂CH₃)O— or —CH₂OCH₂O—;
R^{2b} is hydrogen, hydroxyl, fluoro, alkyl, alkoxy, alkoxyalkoxy, alkylamino, dialkylamino, alkylcarbonylamino, azido, —SH, —CN, —CF₃, —OCF₃, alkyl sulfanylalkoxy, aminooxyalkoxy, alkylaminooxyalkoxy, dialkylaminooxyalkoxy, aminocarbonylalkoxy, alkylaminocarbonylalkoxy or dialkylaminocarbonylalkoxy;
R^{4b} is hydrogen or hydroxyalkyl;
or R^{2b} and R^{4b} together form —CH₂O—, —CH₂NH—, —CH₂S—, —CH₂N(OR^{p})—, —CHCH₃O—, —C(CH₃)₂O—, —CH₂C(═CH)—, —CHCH₃C(═CH)—, —CHCH₃S—, —CH₂NR^{p}—, —CH₂CH₂O—, —CH₂CH₂CH₂O—, —CH₂OCH₂—, —CH(CH₂OCH)O—, —CH(CH₂CH₃)O— or —CH₂OCH₂O—;
R³ is a hydroxyl protecting group or a thiohydroxyl protecting group;
each R^{p} is independently alkyl; and
each Nu is independently a nucleobase.

14. A process according to claim 13, wherein the oligonucleotide comprising a fragment of formula (XIII) is reacted in the presence of a thiooxidation agent or iodine, wherein the concentration of iodine is between about 0.001 M and about 0.01 M, to arrive at an oligonucleotide comprising at least one non-chiral phosphorothioate internucleoside linkage of formula (I) comprising a fragment of formula (XIV) 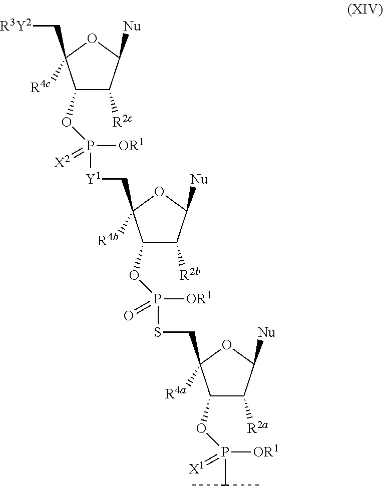
wherein
Y¹ is oxygen or sulfur;
X² is oxygen or sulfur;
provided that Y¹ and X² are not both sulfur at the same time;

15. A process according to claim 14, wherein the oligonucleotide comprising a fragment of formula (XIII) is reacted in the presence of a thiooxidation agent when Y¹ is oxygen.

16. A process according to claim 14, wherein the oligonucleotide comprising a fragment of formula (XIII) is reacted in the presence of iodine when Y¹ is sulfur.

17. A process according to claim 1, wherein the oligonucleotide comprising at least one non-chiral phosphorothioate internucleoside linkage of formula (I) comprises 1 to 8 internucleoside linkages of formula (I).

18. A process according claim 1, wherein the concentration of iodine is between about 0.001 M and about 0.005 M, preferably between about 0.002 M and about 0.005 M.

19. A process according to claim 1, wherein R¹ is cyanoethyl.

20. A process according to claim 1, wherein the hydroxyl protecting group or the thiohydroxyl protecting group is bis-(4-methoxy-phenyl)-phenyl-methyl.

21. A process according to claim 6, wherein R⁵ is diisopropylamino.

22. A process according to claim 1, wherein each Nu is independently selected from adenine, thymine, uracil, guanine and cytosine.

23. A process according to claim 1, wherein the oligonucleotide comprising at least one non-chiral phosphorothioate internucleoside linkage of formula (I) is bound to a solid support for solid phase synthesis.

24. A process according to claim 1, wherein the acid is dichloroacetic acid or trichloroacetic acid.

25. (canceled)

26. (canceled)

27. (canceled)

28. (canceled)

29. A process according to claim 1, wherein the phosphate protecting group R¹ of the oligonucleotide comprising at least one non-chiral phosphorothioate internucleoside linkage of formula (I) is removed to arrive at an oligonucleotide comprising at least one non-chiral phosphorothioate internucleoside linkage of formula (XV)

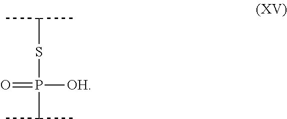

30. (canceled)

31. (canceled)

32. An oligonucleotide manufactured according to a process of claim 1.

33. An oligounucleotide comprising at least one non-chiral phosphorothioate internucleoside linkage of formula (I) 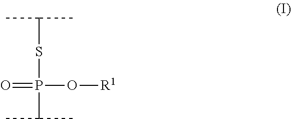
wherein le is a phosphate protecting group
as defined in claim 1 comprising 7 to 31 nucleotides.

34. (canceled)

35. An oligounucleotide comprising at least one non-chiral phosphorothioate internucleoside linkage of formula (I) 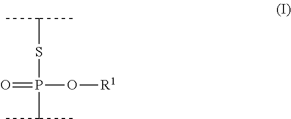
wherein R¹ is a phosphate protecting group
wherein the oligonucleotide comprises at least one nucleotide of formula (XVI) 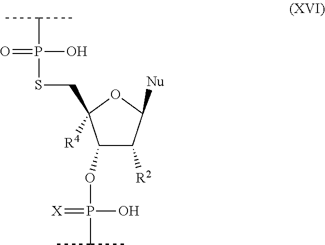
wherein
X is oxygen or sulfur;
R² and R⁴ together form —CH₂O—, —CH₂NH—, —CH₂S—, —CH₂N(OR')—, —CHCH₃O—, —C(CH₃)₂O—, —CH₂C(═CH₂)—, —CHCH₃C(═CH₂)—, —CHCH₃S—, —CH₂NR^{p}—, —CH₂CH₂O—, —CH₂CH₂CH₂O—, —CH₂-O—CH₂—, —CH(CH₂OCH₃)O—, —CH(CH₂CH₃)O— or —CH₂OCH₂O—;
each R^{p} is independently alkyl; and
Nu is a nucleobase.

36. An oligounucleotide comprising at least one non-chiral phosphorothioate internucleoside linkage of formula (I), 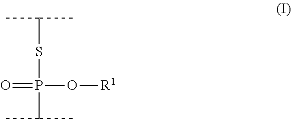
wherein R¹ is a phosphate protecting group
wherein the oligonucleotide comprises at least one nucleotide of formula (XVII) 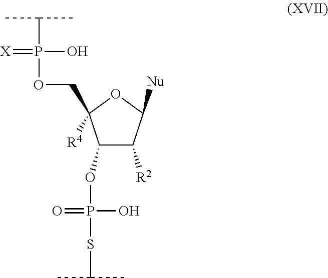
wherein
X is oxygen or sulfur;
R² and R⁴ together form —CH₂O—, —CH₂NH—, —CH₂S—, —CH₂N(OR')—, —CHCH₃O—, C(CH₃)₂O—13 , —CH₂C(═CH₂)—, —CHCH₃C(═CH₂)—, —CHCH₃S—, —CH₂NR^{p}—, CH₂CH₂O—, —CH₂CH₂CH₂O—, —CH₂-O—CH₂—, —CH(CH₂OCH₃)O—, CH(CH₂CH₃)O— or —CH₂OCH₂O—;
each R^{p} is independently alkyl; and
Nu is a nucleobase.

37. (canceled)

38. (canceled)

39. (canceled)

40. (canceled)

41. (canceled)

42. (canceled)

43. (canceled)

44. (canceled)

45. (canceled)
